(19) United States
(12) Patent Application Publication
(10) Pub. No.: US 2021/0277421 A1
(43) Pub. Date: Sep. 9, 2021

(54) Homologous Recombination Reporter Construct and Uses Thereof

(71) Applicant: NemaMetrix, Inc, Eugene, OR (US)

(72) Inventors: Christopher E. Hopkins, Murray, UT (US); Trisha Brock, Eugene, OR (US); Thomas Marshall, Eugene, OR (US); Mary Colasanto, Eugene, OR (US); Zachary Stevenson, Eugene, OR (US)

(73) Assignee: NemaMetrix, Inc, Eugene, OR (US)

(21) Appl. No.: 17/266,592

(22) PCT Filed: Aug. 6, 2019
(86) PCT No.: PCT/US19/45374

§ 371 Date: Feb. 6, 2021

Related U.S. Application Data
(60) Provisional application No. 62/715,117, filed on Aug. 6, 2018.

(51) Int. Cl. C12N 15/90; C12N 9/22; C12N 15/113; C12N 15/82

(52) U.S. Cl. CPC C12N 15/902; C12N 9/22; C12N 2800/80; C12N 15/8212; C12N 15/113

(57) ABSTRACT

The present disclosure provides homologous recombination reporter nucleic acid construct reagents for increasing the likelihood of detecting successful modification of a specific sequence in chromosomal DNA of a host cell via homologous recombination. The homologous recombination reporter constructs contain a sequence element inserted within the coding sequence for a reporter gene resulting in a mutated reporter gene. The sequence element is removed via homologous recombination based on the presence of two homology regions present in the reporter construct.

### CROSS-REFERENCE TO RELATED APPLICATIONS

This application claims the benefit of U.S. Provisional Patent Application No. 62/715,117, filed on 6 Aug. 2018, the content of which is incorporated herein by reference in its entirety.

### STATEMENT REGARDING FEDERALLY SPONSORED RESEARCH OR DEVELOPMENT

This invention was made with government support under R44HD090831 awarded by NATIONAL INSTITUTE OF CHILD HEALTH & HUMAN DEVELOPMENT. The government has certain rights in the invention.

### SEQUENCE LISTING

The instant application contains a Sequence Listing which has been submitted electronically in ASCII format via EFS-Web and hereby incorporated by reference in its entirety. Said ASCII copy, created on 6 Aug. 2019, is named NEMA005PCT_SL_ST25.TXT and is 6283 bytes in size.

### FIELD OF THE INVENTION

This application pertains generally to tools and methods for increasing the likelihood of detecting modification of a specific sequence in chromosomal DNA of a cell via homologous recombination.

### BACKGROUND OF THE INVENTION

Homologous recombination was first demonstrated in 1986, when targeted gene knock-out (KO) of a gene was successfully demonstrated in mice (Thomas, K. R. and Capecchi, M. R. Introduction of homologous DNA sequences into mammalian cells induces mutations in the cognate gene. Nature. 1986 Nov. 6-12; 324(6092):34-8). Knock-in (KI) achievement involving exon swapping occurred 10 year later (Hanks, M., et al. Rescue of the En-1 mutant phenotype by replacement of En-1 with En-2. Science. 1995 Aug. 4; 269(5224):679-82). It was soon discovered that double-strand breaks could enhance gene-targeted transgenesis rates by 100 fold (Rouet, P. et al.). Introduction of double-strand breaks into the genome of mouse cells by expression of a rare-cutting endonuclease. Mol Cell Biol. 1994 December; 14(12):8096-106; Jasin, M., et al. Targeted transgenesis. Proc Natl Acad Sci USA. 1996 Aug. 20; 93(17):8804-8). In regard to gene editing, a synthesis-dependent strand annealing (SDSA) model of homologous recombination became recognized as the dominant mechanism for gene conversion in a recipient chromosome from insertional cargo content of a donor homology plasmid (Pâques, F. and Haber, J. E. Multiple pathways of recombination induced by double-strand breaks in Saccharomyces cerevisiae. Microbiol Mol Biol Rev. 1999 June; 63(2):349-404; Paix, A. et al. Precision genome editing using synthesis-dependent repair of Cas9-induced DNA breaks. Proc Natl Acad Sci USA. 2017 Dec. 12; 114(50): E10745-E10754).

To detect homologous recombination events, the current and most widely used method of detection is based on PCR. A primer annealing to cargo content is paired with a primer that anneals in the genome to a region on the outside of one of the homology arms. PCR product can only occur for events where gene insertion has occurred at the target site. No product is made from either the donor homology plasmid, the unmodified locus, or gene insertion at a non-target site.

Alternative methods to detect gene insertion activity employ expression reporter systems. In zebrafish, the tol2 transposase mediated insertion system typically incorporates a transcriptional GFP reporter (Kwan, K. M. et al. The Tol2kit: a multisite gateway-based construction kit for To12 transposon transgenesis constructs. Dev Dyn. 2007 November; 236(11):3088-99). In mice, the Rosa26 safe harbor locus is typically paired with a floxed GFP reporter (Mao, X., et al.). Activation of EGFP expression by Cre-mediated excision in a new ROSA26 reporter mouse strain. Blood. 2001 Jan. 1; 97(1):324-6.) and transgenic discovery is aided by selection markers such as NeoR as used in embryonic stem cells for site-directed gene insertion with zinc fingers (Landau, D. J. et al. In Vivo Zinc Finger Nuclease-mediated Targeted Integration of a Glucose-6-phosphatase Transgene Promotes Survival in Mice with Glycogen Storage Disease Type IA. Mol Ther. 2016 April; 24(4):697-706), TALENs (Kasparek, P. et al. Efficient gene targeting of the Rosa26 locus in mouse zygotes using TALE nucleases. FEBS Lett. 2014 Nov. 3; 588(21):3982-8) or CRISPR/Cas9 systems (Quadros, R. M., Harms, D. W., Ohtsuka, M. & Gurumurthy, C. B. Insertion of sequences at the original provirus integration site of mouse ROSA26 locus using the CRISPR/Cas9 system. FEBS Open Bio. 2015 Mar. 10; 5:191-7).

Unlike embryonic stem cell approaches, transgenesis occurring directly in a fertilized embryo is a desirable approach to more rapidly derive a transgenic animal. Knock-out driven by non-homologous end joining (NEHJ) repair can occur with sufficient efficiency that a brood of 300 injected embryos will yield progeny with germline edits. For knock-in (KI) transgenes the efficiency is much lower and many more embryos must be screened to find the few animals with trans-generation germline edits. PCR methods for detecting edited animals become a drawback to detect Homologous Recombination (HR)-dependent embryo transgenesis due to the requirement to harvest a biopsy of tissue or cells. Since recovery of the animal is necessary, the injected embryo must be grown to an age that is tolerant of biopsy. Typically, biopsies are obtained weeks to months after hatching of live animals. For many organisms the result is a high vivarium cost from raising large cohorts of animals to biopsy age. Drug resistance selection systems have not yet been deployed to aid in selecting embryos early after injection. Use of fluorescent reporters is typically hampered by high levels of ectopic expression from the plasmid used in transgenesis.

In C. elegans, a co-CRISPR strategy is employed for detecting target site edits by selecting for a second site edit that gives a visible phenotype (Kim, H. et al. A co-CRISPR strategy for efficient genome editing in Caenorhabditis elegans. Genetics. 2014 August; 197(4):1069-80). Other reporters have been developed such as the Traffic-Light reporter (Certo, M. T. et al. Tracking genome engineering outcome at individual DNA breakpoints. Nat Methods. 2011 Jul. 10; 8(8):671-6; Liu, J. et al. Development of novel visual-plus quantitative analysis systems for studying DNA double-strand break repairs in zebrafish. J Genet Genomics. 2012 Sep. 20; 39(9):489-502; Kuhar, R. et al. Novel fluorescent genome editing reporters for monitoring DNA repair pathway utilization at endonuclease-induced breaks. Nucleic Acids Res. 2014 January; 42(1); Wang, L. et al. Simultaneous screening and validation of effective zinc finger nucleases in yeast. PLoS One. 2013 May 31; 8(5); Li, G. et al. Suppressing Ku70/Ku80 expression elevates homology-directed repair efficiency in primary fibroblasts. Int J Biochem Cell Biol. 2018 Apr. 12; 99:154-160; Kan, Y. et al. Mechanisms of precise genome editing using oligonucleotide donors. Genome Res. 2017 July; 27(7):1099-1111) which proved an activated-fluorescence readout of homologous recombination activity. However, the Traffic-Light reporter does not provide self-contained donor homology arms and requires a separate plasmid containing a repair fragment used to instruct proper recoding repair of the nascent but defective GFP fluorescent protein. Additionally, the Traffic-Light reporter has dependency on a homing mega-nuclease which leads to 6 bp overhangs at the 3′ end that must be edited at lower efficiency by additional activation of mismatch repair systems to enable single-strand-annealing (SSA) gene editing. It does not contain at least one perfectly complementary strand for direct 3′ end synthesis after strand invasion into the recipient donor homology. As such, there is a definite gap in the available technology. Gene disruption and new content insertion are valuable tools scientists can use to study gene function and develop new therapeutic discovery platforms.

CRISPR/Cas9 systems are now routine in zebrafish using Cas9-mediated transgenesis (Hwang, W. Y. et al. Efficient genome editing in zebrafish using a CRISPR-Cas system. Nat Biotechnol. 2013 March; 31(3):227-9). For example, a set of Cas9 guide RNAs are selected and tested for their capacity to activate Cas9 nuclease towards high cutting efficiency. A guide is considered to have sufficient efficiency when more than 50% of the injected animals test positive for having a detectable level of imperfect repair at a cut site. Yet current bioinformatics tools predict high efficiency sgRNAs only about half the time. As a result, a set of 3 to 4 sgRNAs is frequently made to a target locus to ensure at least one high efficiency cutter will be discovered and be used to recover frameshifting (insertion/deletion) indels that KO gene function. Yet even with a high efficiency cutter the ability to get precision editing of the genome using a donor homology DNA sequence is a daunting task.

Non-homologous end joining (NHEJ) can be used to obtain in frame insertion of GFP on the C-terminus of various genes (Auer, T. O et al. Highly efficient CRISPR/Cas9-mediated knock-in in zebrafish by homology-independent DNA repair. Genome Res. 2014 January; 24(1):142-53). Using a sgRNA giving 66% mutagenesis in soma, a wide variability in success rate was observed. Germline integration for desired edit is reported to range from 1.2% to 34%. Even with HR repair the high incidence of additional random mutations included with the intended genome modification are observed which effectively pushes the germline efficiency into the single digits (Hwang, W. Y. et al. Efficient genome editing in zebrafish using a CRISPR-Cas system. Development. 2013 December; 140(24):4982-7). One approach to deal the prevalence of (insertion/deletion) indels flanking insertion of large content is an intron-mediated strategy for GFP reporter insertion (Li, J., et al. Intron-based genomic editing: a highly efficient method for generating knockin zebrafish. Oncotarget. 2015 Jul. 20; 6(20):17891-4). It was found the NHEJ mediated approach provided GFP tagging at a native locus at a mean germline transmission rate near 12%. This compared favorably to a tabulation of plasmid-based NHEJ insertion methods which are reported to average 8.5% (Li, J. et al. Intron targeting-mediated and endogenous gene integrity-maintaining knockin in zebrafish using the CRISPR/Cas9 system. Cell Res. 2015 May; 25(5):634-7; Kimura, Y., Hisano, Y., Kawahara, A. & Higashijima, S. Efficient generation of knock-in transgenic zebrafish carrying reporter/driver genes by CRISPR/Cas9-mediated genome engineering. Sci Rep. 2014 Oct. 8; 4:6545; Hisano, Y. et al. Precise in-frame integration of exogenous DNA mediated by CRISPR/Cas9 system in zebrafish. Sci Rep. 2015 Mar. 5; 5:8841). Intron and plasmid based methods utilizing NHEJ are in contrast to HR methods which insert GFP only at an observed 1.5% germline transmission efficiency (Zu, Y. et al. TALEN-mediated precise genome modification by homologous recombination in zebrafish. Nat Methods. 2013 April; 10(4):329-31). Yet although NHEJ methods insert content with higher efficiency, they tend to be highly error prone. Thus, despite the low efficiency with HR methods, getting precise genome editing is still quite attractive for many projects, so there is a need to develop a more generalizable approach to achieve more efficient HR transgenesis methods.

Recent publications are breaking this barrier towards an efficient and generalizable HR approach. Groups using either plasmid based (Trion, U., Krauss, J. & Nüsslein-Volhard, C. Precise and efficient genome editing in zebrafish using the CRISPR/Cas9 system. Development. 2014 December; 141(24):4827-30) or oligonucleotide based approaches (Armstrong, G. A. et al. Homology Directed Knockin of Point Mutations in the Zebrafish tardbp and fus Genes in ALS Using the CRISPR/Cas9 System. PLoS One. 2016 Mar. 1; 11(3); Gagnon, J. A. et al. Efficient mutagenesis by Cas9 protein-mediated oligonucleotide insertion and large-scale assessment of single-guide RNAs. PLoS One. 2014 May 29; 9(5)) are starting to achieve production of perfect edits but the efficiencies are low. Most notable is a double-cutting transgenesis methodology enabling observation of 3 precise germline integrations out of 529 embryos injected (Hisano, Y. et al. Precise in-frame integration of exogenous DNA mediated by CRISPR/Cas9 system in zebrafish. Sci Rep. 2015 Mar. 5; 5:8841). As a result, even though a moderately good sgRNA efficiency is being used, observed efficiency for heritable KI integration of precise in-frame GFP at the C terminus of Krtt1c19e in zebrafish is only 0.57%. Yet encouragingly undesirable indels at the insertion junctions were not observed. In the double-cutting method, both a genomic locus and the donor transgenesis plasmid are cut, each with a separate sgRNA for targeting Cas9 nuclease. Each guide exceeds 50% cutting efficiency for generating KOs. The use of efficient sgRNAs to cut the genome and a donor homology plasmid is necessary to enabled HR induction of a KI edit. The sgRNA was found capable of generating 77% KO mutagenized embryos. The plasmid sgRNA site used an sgRNA for an eGFP sequence that was capable of attaining 66% mutagenized embryos. When both cutters were included with a mixture containing Cas9+ donor homology, the screens of the injections exhibited nearly 1% of the injected embryos as having the desired edit content of interest.

A reporter with strong soma signal can forecast efficiency. Prior work indicates near 1% germline GFP insertion efficiency is foreshadowed by looking in juveniles for GFP fluorescence events in the soma (Hisano, Y. et al. Precise in-frame integration of exogenous DNA mediated by CRISPR/Cas9 system in zebrafish. Sci Rep. 2015 Mar. 5; 5:8841). Soma expression was segregated into three categories (broad, intermediate, narrow). The broad GFP expression category forecasts germline events with the highest precision. 15 animals with broad expression contained 2 of the germline edits out of a total of 529 injected embryos. By limiting the examination to the 15 animals, the theoretical reduction in screening effort is a 35.3× decrease (529/15)−over 97% of the injected animals could have been discarded from any further downstream work. On the intermediate category, the enrichment was not as good. 71 animals showed intermediate expression from which one animal had a desired germline edit (enrichment ratio 529/71=7.45×). Screening applied to the 115 narrow expression animals would not have been useful as no germline edits were detected in this group. All combined, a binary screen choice for GFP had 201 animals out of 529 injected (38%) as positive in the soma for recombination activity. Restricting the screen to the brightest GFP signal from the 15 animals with broad expression, would have detected the majority of the germline edits by examining only 2.8% of the injected. As a result, by letting only bright soma GFP embryos go forward, there would have been a dramatic reduction in animal husbandry and F1 adult screening efforts.

Discovery of germline edits is made easier by restricting embryo selection to bright GFP in soma. For the three germline integrants found in the 15 animals with broad expression (Hisano, Y. et al. Precise in-frame integration of exogenous DNA mediated by CRISPR/Cas9 system in zebrafish. Sci Rep. 2015 Mar. 5; 5:8841), the 2 germline edits present in the broad GFP expression pool were much easier to find. This contrasted to the one edit found in the intermediate GFP expression pool which gave a much lower number of germline edited progeny. The fraction of GFP-positive Fl progeny derived from each of the two broad GFP expression founders was 49.5% and 25.3%. The intermediate GFP founders gave only 2.4% as GFP-positive F1s. As a result, labor effort to find germline edits in the intermediate GFP pool was 15× larger. As a result, by restricting screening efforts to only the broad expression GFP pool, the labor effort in discovering germline alleles would be much lower.

The use of a GFP reporter inserted at the site of editing (Hisano, Y. et al. Precise in-frame integration of exogenous DNA mediated by CRISPR/Cas9 system in zebrafish. Sci Rep. 2015 Mar. 5; 5:8841)) is only useful for inserting GFP at targeted sites in the genome. It is not practical to use GFP insertions as a marker of second site gene edits. The GFP insertion is a permanent marker and would require outcrossing to remove or could be quite difficult to remove if a second-site target edit and the GFP insertion site are linked on the same chromosome. The ideal reporter for identifying recombination competent injections is transient and epigenetic. It should detect recombination events with gradation of response. If a reporter with these properties can efficiently detect recombination-competent embryos, its application in a KI transgenesis procedure would greatly simplify the process of isolating germline alleles in zebrafish.

It has been noted that biallelic transgenesis of germline in F0 injected embryos is very important to achieve and would enable a significant reduction in the husbandry burden for obtaining a homozygous fish (Jao, L. E., Wente, S. R. & Chen, W. Efficient multiplex biallelic zebrafish genome editing using a CRISPR nuclease system. Proc Natl Acad Sci USA. 2013 Aug. 20; 110(34):13904-9). Using the various target genes including the tyr gene sgRNA (GGACTGGAGGACTTCTGGGG), 100% biallele conversion in soma of injected F0s was observed. This then enabled 100% biallelic germline recovery in F1 progeny. As a result, if conditions can be found to efficiently create KI embryos by HR, biallelic conversion of a locus becomes a distinct possibility in the F1 transgenics.

Discovery of editing events early, just after injection, are advantageous because they allow a significant reduction in the size of the pool of animals needing to be raised to an age tolerant of biopsy. Methods that enrich populations for the desired edit prior to typical biopsy age has the potential to provide significant vivarium cost savings in labor and facility usage. A system that can identify HR competent injection within a few days after injection is needed.

### SUMMARY OF THE INVENTION

Herein are provided homologous recombination reporter constructs (e.g., HR reporter construct) and methods of use for increasing the likelihood of detecting successful modification of a specific sequence in chromosomal DNA of a host cell. In certain embodiments, the HR reporter constructs may also be used in methods for identifying test compounds that increase homologous recombination.

In certain embodiments is provided a nucleic acid construct comprising a gene for a mutated fluorescent protein, wherein the gene comprises; a sequence element that disrupts expression of a functional fluorescent protein and wherein the sequence element is removed with successful homologous recombination in a host cell restoring the functional fluorescent protein, wherein the sequence element comprises; a B segment and an A′ segment, wherein the B segment comprises an expression disruption site; and, the A′ segment comprises a direct repeat of an A segment immediately upstream of the B segment, wherein the A segment comprises a portion of a coding sequence of the fluorescent protein from 15 base pairs to 3000 base pairs in length.

In certain embodiments is provided a method for increasing likelihood of detecting successful modification of a specific sequence in chromosomal DNA of a host cell comprising introducing a present HR reporter construct to a host cell, introducing gene editing reagents into the host cell comprising a donor target sequence; and, observing a desired detectable marker expressed from the construct in those host cells with successful homologous recombination gene editing.

In certain other embodiments is provided a method using CRISPR gene editing reagents for increasing likelihood of detecting successful modification of a specific sequence in chromosomal DNA of a host cell via homologous recombination. In embodiments, the methods comprise introducing a present HR reporter construct to a host cell, introducing gene editing reagents into the host cell comprising: Cas9 complexed with a sgRNA that binds a sgRNA recognition site on the construct; Cas9 complexed with a sgRNA that binds a sgRNA recognition site on the chromosomal DNA; and, a genomic insertion sequence located between two homology regions that are homologous with a region of the chromosomal DNA of the host cell; and, observing a desired detectable marker expressed from the construct in those host cells with successful homologous recombination gene editing.

In certain embodiments is provided a method for identifying test compounds that increases homologous recombination in a host cell, wherein the methods comprise introducing a present HR reporter construct into the host cell introducing gene editing reagents into the host cell; introducing a test compounds into the host cell; observing a desired detectable marker expressed from the construct in those host cells with successful homologous recombination gene editing; comparing the desired detectable signal to a control wherein the control is a host cell without a test compound and selecting those test compounds that produced an increased detectable signal in a host cell as compared to the control. In certain embodiments, a test compound is selected from a therapeutic agent, a drug, a drug candidate, a nutritional supplemental, vitamin or food stuff.

In further embodiments are provided an expression plasmid comprising SEQ ID NO. 1, SEQ ID NO. 2, SEQ ID NO. 3 or SEQ ID NO 4. In certain embodiments provided herein is use of a promoter for expression of a gene in a zebrafish embryo, comprising contacting the zebrafish embryo with an expression vector comprising the promoter rpl13a. In embodiments, that reporter is provided as SEQ ID NO. 4.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1A shows conceptualization of a present construct with a sequence coding for a mutant fluorescent protein that comprises a sequence element disrupting expression. Removal of that sequence element via successful homologous recombination results in expression of a functional fluorescent protein. Conceptually, a coding sequence for a fluorescent protein is interrupted by an intervening sequence “B” segment that alters and/or inactivates the fluorescent protein because it comprises an expression disruption site, such as a stop codon. As a homologous recombination reagent, the sequence element further comprises an A′ segment, which is a direct repeat of a coding sequence directly upstream of the B segment (an A segment). Nuclease cleavage and activation of homology-mediated repair enables reconfiguration via homologous recombination into an active expressed fluorescent protein.

FIG. 1B shows conceptual preparation of a present construct wherein an A segment is identified within the coding sequence of a fluorescent protein, a sequence element comprising a B segment and an A′ segment (a direct repeat of the A segment) is inserted directly downstream of the A segment. The B segment comprises an expression disruption site resulting in translation of a mutant fluorescent protein. sgRNA recognition sites flank the A and B interface, and at the B and A′ interface, enable Cas9 homologous recombination resulting in expression of a functional fluorescent protein.

FIG. 2 shows three HR reporter constructs that were made and optimized for use in C. elegans: the pNU751g construct contains long 528 bp homology arms (A and A′ segments) and only one cleavage site with a GFP expression cassette for germline expression; pNU751k is similar to pNU751g but contains two sgRNA sites and a noncoding stuffer DNA in the B segment; and, the pNU924 construct has 41 bp homology arms (A and A′ segments) and avoids transcript run-on and misfolded protein production by fusing upstream of the mCherry segment to inactive GFP coding sequence in frame. The B segments each contain a stop codon that truncates expression of the mCherry fluorescent protein. The construct pNU344 is a control reporter and contains an active fluorescent protein without homologous recombination. The construct comprises the promoter eft-3p and the coding sequence for the mCherry fluorescent protein.

FIG. 3A shows results from C. elegans injections with the construct, pNU751g; FIG. 3B with pNU751k; and, FIG. 3C with the construct pNU924. For each injection, a mixture was made of a construct with Cas9 and appropriate sgRNAs and an oligonucleotide “roller” template targeting the dpy-10 native locus. After injection, the progeny of the injected animal was scored for the presence of red fluorescence at day 1 and day 5. The roller phenotype in the progeny is scored on day 5. An enrichment ratio was calculated as a number of red “hits” relative to total number of injected animals screened. The plasmid pNU924 showed the highest enrichment ratio at day 1. All other configurations either did not develop within 24 hrs (“nd”) or had a lower enrichment ratio. Capture efficiency is percentage of edits captured in red hits. The plasmids pNU924 and pNU751g both showed perfect capture on day 5.

FIG. 4 shows the homologous recombination (HR) reporter increases identification of CRISPR mediated homologous recombination. FIG. 4A. Injections were performed with the reporter plasmid indicated or no reporter plasmid along with CRISPR components for dpy-10 target site mutagenesis. Plates with red fluorescence were counted and divided over the total number of plates. The result is a ratio giving the percent of plates needed to be screened. Efficiency score was generated by subtraction of 1 minus the ratio (1-red/total). Results are an average of 3-4 experiments. FIG. 4B. Representative sequencing results for the dpy-10 locus. Targeted mutation shows the differences from wild-type in bold. Three insertion/deletion (indel) examples are given. Deleted bases are indicated by a “−”. Use of the HR reporter leads to a 27% boost in the isolation of desired edits relative to negative control reaction with no marker. When an always-on red reporter is used with a control plasmid, red selection leads to enrichment of number of edits. When the HR reporter is used in place of control plasmid a similar enrichment is seen but the types of desired mutations (HR Repair) see a boost near 50% while the undesired indels drop by nearly half.

FIG. 5 shows representations of the different Zebrafish reporter constructs used in the examples. The reporter plasmids are the conceptualization of a present plasmid with a sequence coding for a mutant fluorescent protein and that comprises a sequence element disrupting expression comprising a B segment and an A′ segment. A double strand break in expression of the construct that is repaired by homologous recombination leads to a functional fluorescent protein in the different Zebrafish HR reporter configurations. The exemplified present plasmids pNU1597, pNU1455, pNU1902 and pNU1903 were made and optimized for use in Zebrafish. The construct pNU1279 is a control reporter and contains an active fluorescent protein without homologous recombination. The construct comprises the promoter rpl13a and the coding sequence for the mCherry fluorescent protein.

FIG. 6 shows expression of the pNU1279 construct with the promoter rpl13a and the coding sequence for the mCherry fluorescent protein at 8 hours (Panel A and D); 12 hours (Panel B and E); and, 24 hours (Panel C and F) post fertilization in Zebrafish embryos. Panel A, B, and C are bright field images Panel D, E, and F are fluorescent images.

FIG. 7 shows expression of red fluorescent protein in Zebrafish after successful homologous recombination using CRISPR/cas9 genome editing and the pNU1455 construct. B shows an image of expression of red fluorescence protein taken 24 hours after injection wherein red fluorescence is visible in a subset of cells marked by arrow. C shows a PCR assay demonstrating recombination wherein lanes marked 1, 2, 3 are injected embryos; the lane marked plasmid is the un-recombined plasmid control; and, recombination bands are marked with black arrows in lanes 1 and 3. D shows PCR assay demonstrating recombination is dependent on the presence of Cas9 in the injection mix at 24 hours post injection. Injections absent of Cas9 nucleases show no bandshift product. Injections with Cas9 have 6 of 9 embryos exhibiting bandshift.

FIG. 8 shows editing of the native tyrosinase genomic locus. Genetic sequence of the tyrosinase edit (top) and the wild -type tyrosinase (bottom) is seen in top panel A. A three frame stop sequence is added in the edit of tyrosinase. A PCR primer (6566) was designed to amplify from only the edited tyrosinase locus. Agarose gel showing the amplification with the primer specific to the edit is seen in bottom panel B. A band of 631 bp will be present when the edit has occurred. PCR products from 8 embryos injected with the tyrosinase editing CRISPR components are on the left. Seven of these eight show a band of the correct size indicating that editing has occurred. On the right are PCR results from 8 un-injected embryos. None of these contain the band of the correct size because these are unedited.

FIG. 9 shows expression of red fluorescent protein in Zebrafish after successful homologous recombination using CRISPR/cas9 genome editing using the pNU1579 reporter construct. Also pictured are embryos with a dim signal for reference.

FIG. 10 shows expression of red fluorescent protein (mCherry) in Zebrafish embryos as compared to fluorescein. Shown are results from different injection mixes comprising a present HR reporter construct, but no target sgRNA sequence or target repair DNA sequence (Injection Mix 1); present HR reporter construct, genome targeting sgRNA sequence and genome target repair DNA sequence (Injection Mix 2); and, present HR reporter construct, genome targeting sgRNA sequence, genome target repair DNA sequence and p53 morpholino (Injection Mix 3). The results show that p53 morpholino knock down of Zebrafish p53 expression, to reduce in p53-mediated induction of NHEJ activity and increase HR, resulted in an increase in HR reporter activity.

### DETAILED DESCRIPTION OF THE INVENTION

Introduction

The present invention provides methods and compositions for increasing the likelihood of detecting successful modification of a specific sequence in chromosomal DNA of a cell via homologous recombination. In embodiments, the present compositions, which undergo homologous recombination to provide an observable fluorescent signal, are surrogates (or correlate) for a second (also referred to herein as the “genomic insertion sequence”) homologous recombination event that modifies a specific sequence in chromosomal DNA of a cell. In embodiments, the present compositions comprise nucleic acid constructs comprising a sequence for a fluorescent protein that has been mutated by insertion of a sequence element disrupting expression of the fluorescent protein. Those constructs are also referred to herein as homologous recombination (HR) reporter constructs. The present HR reporters or reporter constructs, upon successful homologous recombination, are restored to their non-mutated form, yielding a desired detectable signal, and thus allowing for early detection of successful gene-editing of the chromosomal DNA gene.

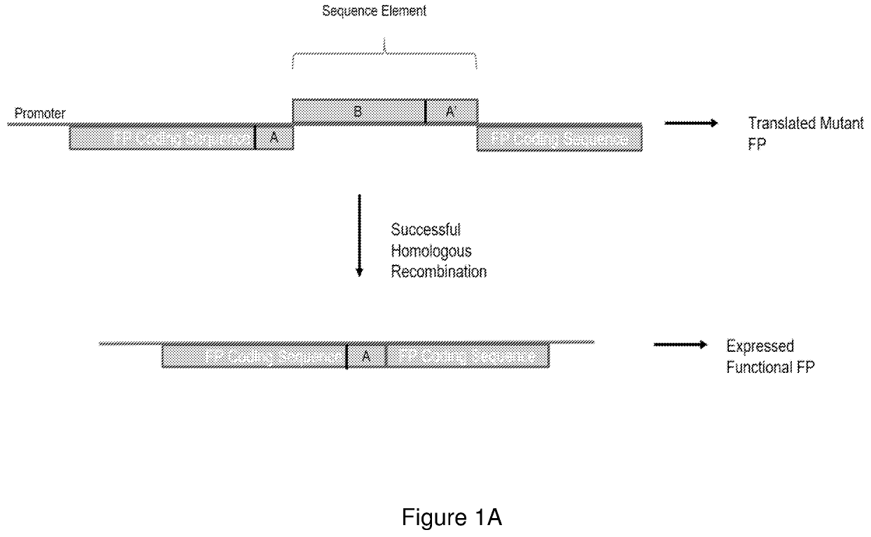

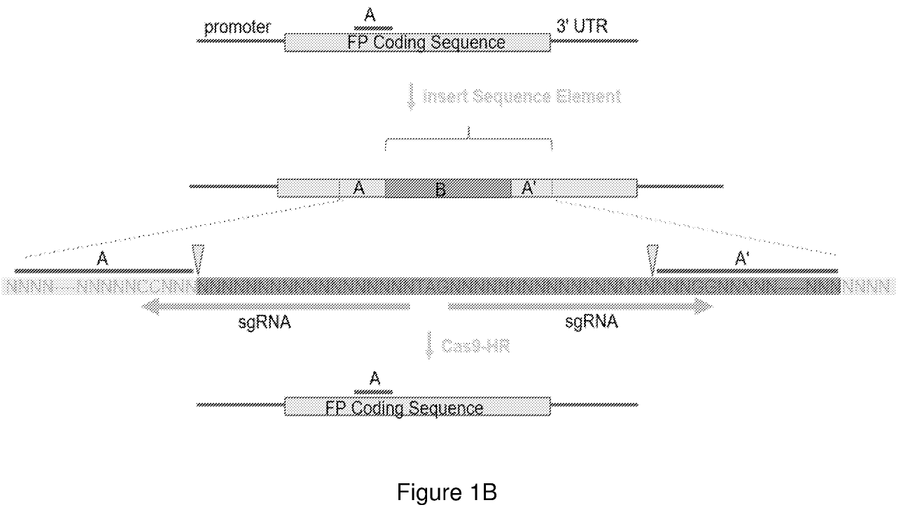

In embodiments, the compositions comprise nucleic acid plasmid constructs that comprise a gene (e.g. coding sequence) of a fluorescent protein that has been mutated by the insertion of a sequence element, wherein translation of that mutated fluorescent protein results in a protein that is either inactive (e.g., little to no detectable fluorescent signal) or qualitatively different (e.g., different color (the emission wavelength is shifted)) as compared to the non-mutated fluorescent protein. The present constructs are configured to restore the activity of the non-mutated fluorescent protein following successful homologous recombination. Constructs were configured and prepared wherein a sequence element was inserted into the coding sequence of the fluorescent protein, and which served both to disrupt expression of the fluorescent protein and enable homologous recombination to remove the sequence element. See FIGS. 1A and 1B.

The present sequence element comprises a B segment and an A′ segment. The A′ segment is a direct repeat of an A segment in the fluorescent protein coding sequence. The B segment and A′ segment are synthesized as the sequence element and inserted directly downstream of the A segment providing a construct comprising an A-B-A′ sequence configuration. The B segment comprises at least one expression disruption site, such as a stop codon, that results in truncated translation of the mutated fluorescent protein. When added to cells, along with homologous recombination reagents, the sequence element is removed via successful homologous recombination (HR) restoring the fluorescent protein to the non-mutated form. The resulting observable signal indicates homologous recombination and is a surrogate or correlate for a second (genomic insertion sequence) homologous recombination event for a successful modification of a specific sequence in chromosomal DNA of a cell via homologous recombination.

In embodiments, the homologous recombination is endonuclease mediated. In certain embodiments, homologous recombination utilizes CRISPR based gene editing reagents, such as Cas9/sgRNA. In that instance, the present HR reporter constructs comprise one or more sgRNA recognition sites. In exemplary embodiments, those sgRNA recognition sites are located near or overlapping the flanking sequence of the A and B interface, or the B and A′ interface, wherein the endonuclease cleaves the sequence between those segments, or one to a few base pairs into those segments. See FIGS. 1B, 2, 5; and, Examples 1 and 4.

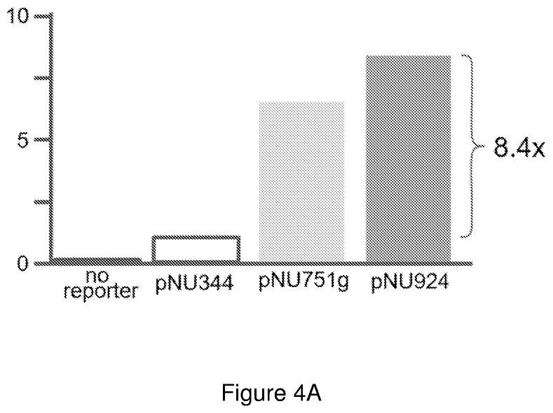

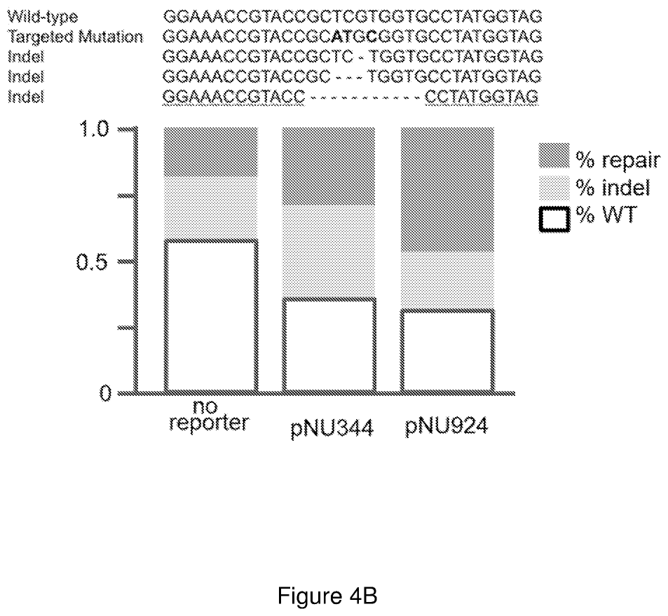

In the context of gene editing, high quality injections occur only at a frequency of 1 or 2 in 200 injections. In other words, the rate of successful homologous recombination following injection of reagents is very low (e.g., 1% or less). Traditionally, confirmation of successful injections (e.g., activation of homologous recombination) requires tissue biopsy performed at a stable post-birth state near adulthood. This creates a high level of expense when 200 animals must be raised and screened to find the one or two animals that contain the desired genome edit. An immense amount of effort is wasted chasing low-quality injections that ultimately do not yield the desired result. In contrast, the use of the present compositions, which are an easily observable surrogate for the genome edit, can reduce the husbandry and screening burden by 20× or more. The data described herein indicates use of the present HR reporter constructs is capable of enriching successful transgenesis injections by 7-fold or more. See FIG. 4.

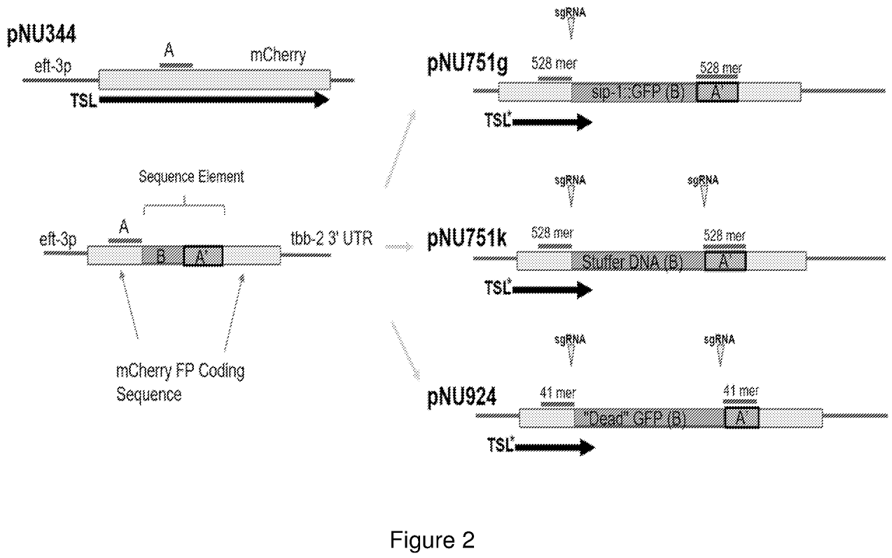

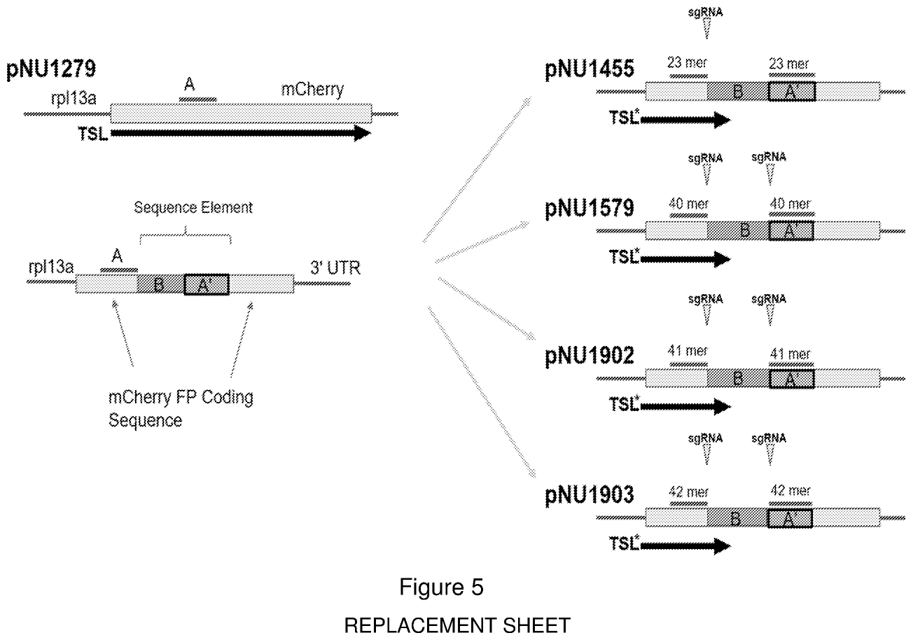

In embodiments, the present HR reporter constructs are configured to restore fluorescence when homologous recombination repair mechanisms have been activated in an embryo and successful gene-editing has occurred. In embodiments, a present HR reporter construct (e.g., those of FIG. 2 or FIG. 5) is introduced into the cell or embryo, wherein the presence of the sequence element in the coding sequence of the fluorescent protein results in an altered or inactive translated fluorescent protein. The B segment of the sequence element comprises all or part of at least one sgRNA recognition site. In embodiments, cleavage of the sgRNA recognition site with a Cas9/sgRNA complex enables repair of the reporter construct to proceed by either NHEJ (non-homologous end joining) or HR (homologous recombination). In NHEJ mediated repair, the cut ends re-ligate into forms unproductive (e.g., not fluorescent). However, if HR repair has been activated, the homology of the direct repeats instructs perfect repair and an active fluorescent protein is produced wherein the sequence element is removed. Therefore, homologous recombination activity in an injection is detected as a burst of fluorescent protein production that is a surrogate detectable signal for successful homologous recombination of the target genomic site.

In embodiments, various disrupted detectable marker genes and/or protein can be used for preparation of the present HR reporter constructs. One example of a disrupted detectable marker is a gene encoding a fluorescent protein that is modified so that the full-length protein is not produced. In certain embodiments, the HR reporter plasmid may be configured wherein, the sequence element comprising a stop codon, a degron signal, rare codon, an RNA splice donor signal, a self-cleaving peptide site, or inactivating point mutations, resulting in a translated fluorescent protein that is truncated producing little to no observable fluorescence. In certain other embodiments, the HR reporter plasmid is configured to comprise a second coding sequence for a fluorescent protein nested between the A segment and A′ segment. See pNU751g of FIG. 2. In embodiments, the B segment comprises a coding sequence for the second detectable marker such that the disrupted reporter gene turns on and the second detectable marker turns off upon successful homologous recombination.

The activity of the disrupted detectable marker can be restored following injection into a cell if a successful gene editing event occurs when used in conjunction with appropriate reagents (e.g. Cas9/sgRNA complex). In certain embodiments, the present HR reporter construct also comprises one or more CRISPR sgRNA sites. The present HR constructs further comprise an appropriate promoter to drive expression of the construct upon introduction to a target cell (e.g., embryo) and a termination signal (3′ UTR).

Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

As used herein, the term “homology driven recombination” or “homology directed repair” or “HDR” is used to refer to a homologous recombination event that is initiated by the presence of double strand breaks (DSBs) in DNA (Liang et al. 1998); and the specificity of HDR can be controlled when combined with any genome editing technique known to create highly efficient and targeted double strand breaks and allows for precise editing of the genome of the targeted cell; e.g. the CRISPR/Cas9 system (Findlay et al. 2014; Mali et al. February 2014; and Ran et al. 2013).

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

As used herein, the term “polynucleotide” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA as DNA construct, mRNA, and anti-sense RNA, any of which can be single stranded or double stranded. The terms “polynucleotide,” “nucleotide sequence” “nucleic acid,” “nucleic acid molecule,” and “oligonucleotide” are also used interchangeably herein to refer to a heteropolymer of nucleotides. Except as otherwise indicated, nucleic acid molecules and/or polynucleotides provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

The terms “transformation,” “transfection,” and “transduction” as used interchangeably herein refer to the introduction of a heterologous nucleic acid into a cell. Such introduction into a cell may be stable or transient. Thus, in some embodiments, a host cell or host organism is stably transformed with a polynucleotide of the invention. In other embodiments, a host cell or host organism is transiently transformed with a polynucleotide of the invention. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromasomally, for example, as a mini-chromosome or a plasmid. In certain embodiments, the nucleotide sequences, constructs, expression cassettes can be expressed transiently and/or they can be stably incorporated into the genome of the host organism, such as in a native, non-native locus or safe harbor location.

As used herein, the term “nematode” refers to an organism that is a member of the phylum Nematoda, commonly referred to as roundworms. Nematodes include free-living species (such as the soil nematode C. elegans) and parasitic species. Species parasitic on humans include ascarids, filarias, hookworms, pinworms, and whipworms. It is estimated that more than two billion people worldwide are infected with at least one nematode species. Parasitic nematodes also infect companion animals and livestock, including dogs and cats (e.g., Dirofilaria immitis; heartworm), pigs (Trichinella spiralis), and sheep (e.g., Haemonchus contortus). There are also nematode species which are parasitic on insects and plants.

As used herein, the term “surrogate” refers to a homologous recombination event (i.e., HR reporter construct) that produces an observable signal correlated to a second homologous recombination event of genomic DNA that does not produce an observable signal.

All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Homologous Recombination Reporter Constructs

Provided herein are compositions and methods that provide a visual indicator of successful modification of a specific sequence in chromosomal DNA of a cell via homologous recombination. The methods utilize a present homologous recombination (HR) reporter construct and a donor homology template (e.g., genome insertion sequence) for incorporation of a gene edit into the chromosomal DNA. The HR reporter construct is visualized when the construct comprising a mutated reporter gene is repaired via homologous recombination. Activating homologous recombination repair mechanisms of a cell, wherein the reporter construct is activated, increases the likelihood that the donor homology template was also repaired via homologous recombination resulting in modification of a specific sequence in the chromosomal DNA of a cell. The present HR reporter construct is a surrogate for a successful modification of a specific sequence in chromosomal DNA of a cell via homologous recombination and in certain embodiments, is used as a separate reagent, but in combination with, transfection reagents comprising target donor sequences.

In embodiments, identification of successful targeted genome editing via homologous recombination is increased using the present HR reporter constructs. In certain embodiments, increased likelihood that repair of the mutated fluorescent protein of the HR report construct correlates to successful targeted genome editing via homologous recombination of the host cell chromosomal DNA is at least 2×, at least 3×, at least 4×, at least 5×, at least 6×, at least 7×, at least 8×, at least 9×, at least 10×, at least 12×, at least 14×, at least 16×, at least 18×, at least 20×, at least 25× or at least 30×. In embodiments, observation of a fluorescent signal (e.g., use of a present HR reporter construct following successful homologous recombination) indicates a likelihood of a successful second homologous recombination event (e.g. modification of a specific sequence in chromosomal DNA of a host cell) is between 5× and 20×.

In exemplary embodiments, the donor homology template is provided as a separate construct from the HR reporter construct, wherein the reporter protein following homologous recombination to remove the sequence element, is expressed epigenetically. In that instance it is understood the HR reporter construct comprises an appropriate promoter to drive expression and a stop codon. Various promoters can be used in the HR reporter constructs, wherein selection is based on the host animal, development stage and/or tissue expression. Exemplified promoters for the present HR reporter construct include eft-3p for C. elegans and rpl13a for zebrafish. See FIGS. 2 and 5; and Example 1 and 4. It is understood promoters are chosen to successfully drive expression of the elements of the HR reporter construct in the desired organism or cell type.

In certain embodiments, the present HR reporter construct and use thereof find utility in embryos for the preparation of transgenic organisms. In embodiments, the host cell (site of homologous recombination) is an embryonic cell of a fish (e.g., zebrafish, medaka, salmon, carp, gar), a mammal (e.g., mice, rat, hamster, rabbit, chicken, pig, cow, horse, primates, human, sheep), a worm (e.g., nematode, including both standard and parasites), a fly (e.g., drosophila), or an insect (e.g., bees, carnivorous beetles, weevils, mosquito, etc.).

In certain embodiments, the present HR reporter construct and use thereof find utility in screening or identifying compounds that improve or increase HR. In that instance, compounds are identified that can be developed for enhancement of the body's natural repair mechanism, potentially reducing DNA damage that leads to cancer or other diseases.

In embodiments, a genetically encoded reporter specifically activated by homologous recombination (HR reporter construct) can express in an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, or a human cell. In certain embodiments, a HR reporter construct is genomically integrated (e.g. germline), wherein the genetically-encoded reporter specifically activated by homologous recombination can express in an organism selected from the group consisting of: an archaea, a bacterium, a eukaryotic single-cell organism, an algae, a plant, an animal, an invertebrate, a fly, a worm, a cnidarian, a vertebrate, a fish, a frog, a bird, a mammal, an ungulate, a rodent, a rat, a mouse, and a non-human primate. In certain embodiments, transiently-expressed, genetically-encoded reporter specifically activated by HR (HR reporter construct) can express in an organism selected from the group consisting of: an archaea, a bacterium, a eukaryotic single-cell organism, an algae, a plant, an animal, an invertebrate, a fly, a worm, a cnidarian, a vertebrate, a fish, a frog, a bird, a mammal, an ungulate, a rodent, a rat, a mouse, and a non-human primate.

In embodiments are provided nucleic acid constructs comprising a gene for a mutated fluorescent protein, wherein the gene comprises; a sequence element that disrupts expression of a functional fluorescent protein and wherein the sequence element is removed with successful homologous recombination in a host cell resulting in a functional fluorescent protein. In embodiments, the sequence element comprises a B segment and an A′ segment, wherein the B segment comprises an expression disruption site; and, the A′ segment comprises a direct repeat of an A segment immediately upstream of the B segment, wherein the A segment comprises a portion of a coding sequence of the fluorescent protein from 15 base pairs to 3000 base pairs in length. See FIGS. 1A, 1B, 2 and 5.

In embodiments, the sequence element is removed via homologous recombination in a host cell providing a functional fluorescent protein with a desired detectable signal. In embodiments, the reporter protein gene encodes a protein that can be detected spectrophotometrically or visually. In certain embodiments, the desired detectible signal is a qualitative signal, wherein a control produces little or no signal and a successful recombination resulting in a functional fluorescent protein providing a desired detectable signal is qualitatively more than the control signal. In embodiments, any fluorescent signal above, or more than, background (e.g. control) is deemed a desired detectable signal. In embodiments, the desired detectable signal is X1, X2, X3, X5, X10, X15, X20 above background or a negative control. In certain embodiments, the coding sequence for the reporter protein is codon optimized for the host cell. In exemplary embodiments, the host cell is a nematode or zebrafish cell.

In embodiments, the present HR reporter construct comprises a coding sequence for green fluorescent protein (GFP), cyan fluorescent protein (CFP), or red fluorescent protein (RFP) (e.g., mCherry, Tag-RFP, etc.). In alternative embodiments, the HR reporter construct comprises a coding sequence for a detectable reporter such as luciferase, a luminescent reporter (e.g., Ranella, Firefly, etc.). See e.g., Pollock et al., Trends in Cell Biology 9:57 (1999). In embodiments, the coding sequence may code for wild type protein, spectral variants of those proteins which retain the ability to be expressed and fluoresce, fluorescent protein fused to a tag, e.g., his-GFP or his-RFP, which is histone H2B fused to the indicated fluorescent protein.

In embodiments, present HR reporter constructs comprises a coding sequence selected from AcGFP, AcGFP1, AmCyan, AmCyan1, AQ143, AsRed2, Azami Green, Azurite, BFP, Cerulean, CFP, CGFP, Citrine, copGFP, CyPet, dKeima-Tandem, DsRed, dsRed-Express, DsRed-Monomer, DsRed2, dTomato, dTomato-Tandem, EBFP, EBFP2, ECFP, EGFP, Emerald, EosFP, EYFP, GFP, HcRed-Tandem, HcRedl, JRed, Katuska, Kusabira Orange, Kusabira Orange2, mApple, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T-Sapphire, TagCFP, TagGFP, TagRFP, TagRFP-T, TagYFP, tdTomato, Topaz, TurboGFP, Venus, YFP, YPet, ZsGreen, or ZsYellow1, which are described in the literature or otherwise commercially available; hRFP and hsRFP are RFP's fused to e.g., a histone protein like H2B from C. elegans.

In embodiments, the coding sequence for any of the disclosed reporter genes herein may be mutated by insertion of a sequence element within the coding sequence of the reporter. See FIG. 1. In embodiments, the HR reporter construct comprises a sequence element that comprises a B segment and an A′ segment. The A′ segment is a direct repeat of an A segment from the coding sequence of the reporter gene, wherein the sequence element is inserted directly downstream of the A segment resulting in a HR reporter construct comprising an A-B-A′ sequence configuration. The construct further comprises coding sequence for the reporter gene, wherein removal of the sequence element restores the original functional coding sequence for the reporter gene. See FIGS. 1A and 1B.

To ensure the reporter gene mutated with the sequence element is not expressed as a functional fluorescent protein, the sequence element comprises an expression disruption site. In embodiments, the expression disruption site is present in the B segment of the sequence element. In embodiments, the expression disruption site comprises a stop codon, frame shift, a degron signal, an RNA splice donor signal, a self-cleaving peptide or a codon that destabilizes expression. In exemplary embodiments, the B segment comprises a stop codon. In certain embodiments, the expression disruption site may be present anywhere within the B segment. In other certain embodiments, the expression disruption site is present at the 3′ end of the B segment at or near the B segment and A′ segment interface.

The A and A′ segments are the homology arms for homologous recombination, which may be initiated via endonuclease cleavage, or independent of an endonuclease. The A and A′ homology segments may be from 15 base pairs to 3000 base pairs in length, or from 20 base pairs to 1000 base pairs in length. In exemplary embodiments, the A and A′ homology segments are from about 20 base pairs to about 550 base pairs in length.

In certain embodiments, the HR reporter construct comprises one or more nuclease cleavage sites. In embodiments, the nuclease cleavage site is at, or near, the interface between the A and B segments. In other embodiments, the nuclease cleavage site is at, or near, the interface between the B and A′ segments. In exemplary embodiments, the HR reporter construct comprises two nuclease cleavage sites, one located at, or near, the interface between the A and B segments, and a second site at, or near, the interface between the B and A′ segments. The cut site may be one or a few nucleotides within the A segment, provided expression of the reporter protein is restored via removal of the sequence element.

In embodiments, the nuclease cleavage site is recognized by nucleases selected from Cas9, Cas12a, Cpf1, TALen, Zinc-finger, I-Sce I, Endo.sce, HO, I-Ceu I, I-Chu I, I-Cre I, I-Csm I, I-Dir I, I-DMO I, I-Flmu I, I-Flmu II, I-Ppo I, I-Sce III, I-Sce IV, I-Tev I, I-Tev II, I-Tev III, PI-Mle I, PI-Mtu I, PI-Psp I, PI-Tli I, PI-Tli II or PI-Sce V. In certain embodiments, the nuclease is a CRISPR Cas nuclease. In exemplary embodiments, the nuclease is Cas9 complexed with sgRNA, wherein the HR reporter construct comprises one or more sgRNA recognition sites. In embodiments, the sgRNA recognition site is at, or near, the interface between the A and B segments of the HR reporter construct. In other embodiments, the sgRNA recognition site is at, or near, the interface between the B and A′ segments of the HR reporter construct. In exemplary embodiments, the HR reporter construct comprises two sgRNA recognition sites, one located at, or near, the interface between the A and B segments, and a second site at, or near, the interface between the B and A′ segments. The cut site may be present at the repeat sequence junction (e.g. A and B segment junction, or B and A′ segment junction) or occur a few nucleotides within the A segment or A′ segment, provided expression of the reporter protein is restored via removal of the sequence element. See FIG. 1B.

In one embodiment, a HR reporter construct that fluoresces after recombining from one side is provided. According to this embodiment, the HR reporter construct comprises a promoter, a mutated gene encoding a detectable marker (e.g., fluorescent protein) comprising a sequence element, a nuclease cleavage site, and 3′UTR or termination signal, wherein the nuclease cleavage site occurs at or near the interface between the A and B segments of the HR reporter construct. In other embodiments, a HR reporter construct that fluoresces after recombining from either side is provided. According to this embodiment, the HR reporter construct comprises a promoter, a mutated gene encoding a detectable marker (e.g., fluorescent protein) comprising a sequence element, a pair of nuclease cleavage sites, and 3′UTR or termination signal, wherein each nuclease cleavage sites occur in tandem at, or near, the interface been the A and B segments and at, or near, the interface between the B and A′ segments.

In an embodiment, a HR reporter construct that expresses a protein that folds properly yet is fluorescent-inactive then fluoresces after recombining is provided. In another embodiment, a HR reporter construct that expresses a protein that fluoresces red upon recombination and concomitantly loses green fluorescence is provided. In another embodiment, a HR reporter construct comprising sequence synonymous to genome (e.g., CRISPR targeting site) is provided. In another embodiment, a HR reporter construct comprising sequence non-synonymous to genome (e.g., CRISPR targeting site) is provided. This allows for tuning of the efficiency of cutting. In one embodiment, a HR reporter construct that uses Cas9 (or another nuclease including other CRISPR nucleases) cutting of genome to activate repair is provided. In one embodiment, a HR reporter construct that uses Cas9 (or another nuclease) cutting of plasmid to activate repair is provided. In one embodiment, a HR reporter construct that is integrated in a genome is provided.

In embodiments, the HR reporter construct comprises a promoter driving expression of the mutated reporter gene that comprises the two homology regions (A and A′ segments) and a B segment resulting in a non-functional or inactivated fluorescent protein. In embodiments, the HR reporter construct would not express a functional reporter protein (e.g., fluorescent protein) under normal circumstances (e.g., when the desired genome editing event does not occur after injection or transfection). When co-injected (or transfected) with an endonuclease (e.g., Cas9 and the appropriate sgRNA) the HR reporter construct would recombine and create a gene encoding a functioning reporter protein (e.g., functional fluorescent protein coding sequence). The construct, in certain aspects, can also have one or more other functional genes (e.g., encoding other fluorescent protein marker) that are functional when not-recombined. In certain embodiments, the HR reporter construct can be used to co-inject or co-transfect for monitoring CRISPR genome editing events, wherein the HR reporter construct comprises one or more sgRNA recognition sites.

In exemplary embodiments, a HR reporter construct that is transiently expressed epigenetically is provided. An advantage of transient expression is that the HR reporter construct will be lost over time if it is not incorporated in the genome. This implementation can be useful for certain genomic edits, such as KI (knock-in) of fluorescent proteins, wherein the HR reporter construct signal would not interfere over time with the KI signal.

In embodiments, a HR reporter construct is provided for use in injections to detect embryos activated for homologous recombination repair. In embodiments, a HR reporter construct is provided for use in injections to detect animals with successful modification of a specific sequence in chromosomal DNA via homologous recombination. In embodiments, a HR reporter construct is provided for use in injections to detect plants with successful modification of a specific sequence in chromosomal DNA via homologous recombination. In embodiments, a HR reporter construct is provided for use in injections to detect cells with successful modification of a specific sequence in chromosomal DNA via homologous recombination. In embodiments, a HR reporter construct is provided as an integrated cell line to monitor HR frequency in an animal. In certain embodiments, the cell line has an integrated HR reporter construct that is non-fluorescent without recombination. After the genome is cut or edited, the HR reporter construct cell line reports that homologous recombination occurred.

In embodiments, a HR reporter construct is provided as an integrated line to monitor HR frequency in an animal after drug treatment. In certain embodiments, a HR reporter construct comprising ratiometric green fluorescent protein to red fluorescent protein and Cas9 are integrated in the genome. In certain embodiments, Cas9 expression is via an inducible promoter and the sgRNA are provided by feeding. Without induction of Cas9 and sgRNA feeding, and hence endonuclease mediated homologous recombination, the integrated HR report construct expresses a green fluorescent protein (the red fluorescent protein is only expressed following repair via HR). With induction of Cas9 and sgRNA feeding (both are needed) animals will become red, wherein the mutated red fluorescent protein of the HR reporter construct is repaired excising out the green fluorescent protein coding sequence. Measurement of the ratio of green to red signal, is the baseline. Subsequently a drug candidate is added to the system. If there is a change in the ratio of green to red signal the drug candidate has affected the ability of HR repair. HR repair is the error-free native system, while NHEJ is error prone. In certain embodiments, other reporter proteins can be used instead of the red and green fluorescent protein.

In embodiments, a HR reporter construct is provided that is transiently expressed comprising ratiometric green fluorescent protein (GFP) to red fluorescent protein (RFP), wherein the coding sequence for the GFP is nested within the RFP between an A segment and A′ segment of the sequence element. In this instance, an appropriate nuclease (e.g. Cas9 complexed with sgRNA) is added to a host cell along with the HR reporter construct and a drug candidate to be screened. Drug candidates with little to no impact on homologous recombination, or even those that increase homologous recombination mediated repair, will provide a host cell that fluoresces red. In other words, the coding sequence for the GFP is removed via homologous recombination. Alternatively, drug candidates that shift repair away from homologous recombination to error prone NHEJ repair will provide a host cell that fluoresces green. In other words, the coding sequence for the RFP was not removed via homologous recombination and is expressed.

In embodiments, a HR reporter construct acts as a surrogate for Cas9 sgRNA cutting and efficiency; creates a tool to see efficiency of a specific locus sgRNA site. In embodiments, a HR reporter construct is provided for use in injections to detect embryo activated for single strand repair. In another embodiment, a HR reporter construct is provided for use in injections to detect embryo activated for NHEJ repair. In another embodiment, a HR reporter construct is provided for use in injections to detect embryos activated for microhomology repair. In one embodiment a HR reporter construct for highest fluorescence signal correlation with a precise knock-in transgenesis is provided for direct injection into embryos (e.g., eukaryotic).

In exemplary embodiments, a zebrafish-optimized HR reporter construct is provided having a promoter, codon optimized gene encoding a fluorescent protein and intron composition that is capable of providing high fluorescence which correlates with a target edit.

In one embodiment, the HR reporter construct comprises introns sufficient to increase expression (e.g., over constructs without introns). In embodiments, the introns are selected so as to not have cryptic splice junctions or alternatively, prevent designed introns from splicing. In embodiments, the HR reporter constructs comprising of codon-optimized coding sequence for the reporter protein have 3 pairs of introns inserted at between appropriate NAG-GTN coding positions. In embodiments, the introns are selected as the shortest native introns from highly-expressed embryonic genes e.g., ribosomal long and short proteins, tubulins and actins. In embodiments, the sgRNA sites utilize target sequences not present in the target genome but closely matching the consensus sequence for the most optimal cutting.

In embodiments, the HR reporter construct comprises a strong promoter for embryonic expression to drive expression of the reporter gene. The HR reporter constructs when co-injected with appropriate sgRNA and Cas9 nuclease, may provide a detectable reporter signal. In embodiments, the detectable reporter is visible within 1, 2, 3, 4, or 5 days (e.g., less than 48 hr, 36 hr, 24 hr, 18 hr or 12 hr).

In embodiments, the HR reporter construct indicates the likelihood (e.g., via fluorescence) which embryos are receiving the desired targeted chromosomal mutagenesis via homologous recombination. In embodiments, the methods comprise introducing a present HR reporter construct into a host cell; introducing gene editing reagents into the host cell comprising a donor target sequence; and, observing a detectable marker in those host cells with successful gene editing.

In embodiments, the gene editing reagents comprise an endonuclease. In embodiments, the donor target sequence comprises a genomic insertion sequence flanked by homology regions, wherein the regions are homologous with a region of a genome of the host cell. In exemplary embodiments, the gene editing reagents comprise a sgRNA/Cas9 complex wherein the sgRNA binds a sgRNA recognition site on the HR reporter construct. In certain embodiments, the gene editing reagents comprise a sgRNA/Cas9 complex wherein the sgRNA binds a sgRNA recognition site on the chromosomal DNA of the host cell.

In certain embodiments is provided a method of increasing likelihood of detecting successful modification of a specific sequence in chromosomal DNA of a host cell via homologous recombination using CRISPR editing reagents. In embodiments, the methods comprise introducing a present HR reporter construct into a host cell, introducing gene editing reagents into the host cell comprising: Cas9 complexed with a sgRNA that binds a sgRNA recognition site on the construct; Cas9 complexed with a sgRNA that binds a sgRNA recognition site on the chromosomal DNA; and, a genomic insertion sequence located between two homology regions that are homologous with a region of the chromosomal DNA of the host cell; and, observing a desired detectable marker expressed from the construct in those host cells with successful homologous recombination gene editing.

In embodiments, the present HR reporter can be a biosensor in screening for drugs that modulate NHEJ vs HDR activities. It has been demonstrated that knock down NHEJ pathway leads to activation of HDR activity (Aksoy et al., Chemical reprogramming enhances homology-directed genome editing in zebrafish embryos. Commun Biol. 2019 May 23; 2:198. doi: 10.1038/s42003-019-0444-0. eCollection 2019). As a result, the HR reporter can be used with a combination of drugs/compounds that knock down NHEJ pathways and their drug activity is detected as higher HDR activity. The system can be use in transient and integrated modes in a variety of animal models. In the transient mode, the HR reporter doesn't integrate, making the construct useful across a number of systems and organisms without the need to tailor for each organism, for example as a reporter in a variety of biological systems (cell culture, mouse, fish, fly, worm, etc.). Geisinger & Stearns (CRISPR/Cas9 Treatment Causes Extended TP53-Dependent Cell Cycle Arrest in Human Cells. bioRxiv. Posted Apr. 10, 2019 doi: 6045382019) teach that CRISPR/Cas9-mediated cutting induces TP53-dependent cell cycle arrest, which is reminiscent of p53-mediated cell death associated with morpholino technologies (Robu et al., p53 Activation by Knockdown Technologies. PLoS Genet. 2007 May 25; 3(5):e78. Epub 2007 Apr. 10.). This result suggests that it should be possible to create transient knock-down of TP53 using morpholino in zebrafish and enhance germline editing with CRISPR locus-specific targeting.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1

### Preparation of Present Nucleic Acid Construct for Use in C. elegans

Provided herein are exemplary configurations of the present nucleic acid construct comprising a mutant fluorescent protein. See FIG. 2. The nucleic acid constructs prepared herein (HR-reporter) comprise a coding sequence for a fluorescent protein, mCherry in the examples below, that is interrupted by a sequence element that disrupts expression of a functional fluorescent protein and wherein the sequence element is removed with successful genomic gene editing in a host cell resulting in a functional fluorescent protein. The sequence element comprises an A′ segment that is a direct repeat of a coding sequence (A segment) of the fluorescent protein located directly upstream of the sequence element. The sequence element may include sgRNA site(s), that may span into the A and A′ segments. See FIG. 1B. Alternatively the sgRNA sites(s) may be wholly contained in the B segment. Endonuclease cleavage and activation of homology-mediated repair enable reconfiguration in situ into an active fluorescent protein. As a control, a plasmid with an uninterrupted fluorescent mCherry protein under the control of the eft-3 promoter was also created, pNU344. See FIG. 2. Three configurations of the HR-reporter constructs were built, comprising three different sequence elements disrupting the coding sequence for mCherry. As shown in FIG. 2, the pNU751g plasmid has two 528 bp homology repeats (A and A′ segments), one sgRNA cleavage site and a B segment containing a GFP expression cassette for germline expression. The pNU751k plasmid is similar to pNU751g but has two sgRNA sites and a B segment containing noncoding “stuffer” DNA. The pNU924 plasmid has 40 bp repeats (A and A′ segments), two flanking sgRNA sites, and avoids transcript run-on/misfolded protein production by fusing upstream mCherry segment to an inactive “dead” GFP coding sequence in frame (B segment). The constructs were made using standard molecular biology techniques for generating recombinant plasmids. The C. elegans codon-optimized mCherry sequence was used for fluorescent protein reporter activity (SEQ ID NO. 1) and the promoter used was from eft-3 gene (SEQ ID NO. 2) for strong ubiquitous expression. The tbb-2 3′utr was used as a 3′ UTR that is permissive for expression.

SEQ ID NO. 1
ATGGTCTCCAAGGGAGAGGAGGACAACATGGCCATCATCAAGGAGTTCAT
GCGTTTCAAGGTCCACATGGAGGGATCCGTCAACGGACACGAGTTCGAGA
TCGAGGGAGAGGGAGAGGGACGTCCATACGAGGGAACCCAAACCGCCAAG
CTCAAGGTCACCAAGGTAAGTTTAAACATATATATACTAACTAAGGAGGC
CCTGATTATTTAAATTTTCAGGGAGGACCCCTCCCTTTTGCTTGGGATAT
TCTTTCCCCCCAATTCATGTACGGATCTAAAGCCTACGTCAAGCACCCAG
CCGACATCCCAGACTACCTCAAGCTCTCCTTCCCAGAGGGATTCAAGTGG
GAGCGTGTCATGAACTTCGAGGACGGAGGAGTCGTCACCGTCACCCAAGA
CTCCTCCCTCCAAGACGGAGAGTTCATCTATAAGGTAAGTTTAAACAGTT
CGGCGCGCCCTAACCATACATATTTAAATTTTCAGGTCAAGCTCCGTGGA
ACCAACTTCCCATCCGACGGACCAGTCATGCAAAAGAAGACCATGGGATG
GGAGGCCTCCTCCGAGCGTATGTACCCAGAGGACGGAGCCCTCAAGGGAG
AGATCAAGCAACGTCTCAAGCTCAAGGTAAGTTTAAACATGATTTTACTA
ACTAACTAATCTGATTTAAATTTTCAGGACGGAGGACACTACGACGCCGA
GGTCAAGACCACCTACAAGGCCAAGAAGCCAGTCCAACTCCCAGGAGCCT
ACAACGTCAACATCAAGCTCGACATCACCTCCCACAACGAGGACTACACC
ATCGTCGAGCAATACGAGCGTGCCGAGGGACGTCACTCCACCGGAGGAAT
GGACGAGCTCTACAAGTAA
SEQ ID NO. 2
tgtttctgttaaattaatgaatttttcataaaataaagacattatacaat
ataaaaatgaagaatttattgaaaataaactgccagagagaaaaagtatg
caacactcccgccgagagtgtttgaaatggtgtacggtacattttcgtgc
taggagttagatgtgcaggcagcaacgagagggggagagatttttttggg
ccttgtgaaattaacgtgagttttctggtcatctgactaatcatgttggt
tttttgttggtttattttgtttttatctttgtttttatccagattaggaa
atttaaattttatgaatttataatgaggtcaaacattcagtcccagcgtt
tttcctgttctcactgtttagtcgaatttttattttaggctttcaacaaa
tgttctaactgtcttatttgtgacctcactttttatatttttttaatttt
taaaaatattagaagtttctaggataattttttcgacttttattctctct
accgtccgcactcttcttacttttaaattaaattgtttttttttcagttg
ggaaacactttgctcactccgta

### Example 2

### Method of Increasing Likelihood of Detecting Successful Modification of a Specific Sequence in Chromosomal DNA of C. elegans Using CRISPR/Cas9

Nucleic acid constructs were prepared according to Example 1 and used in methods for detecting successful homologous recombination of a target sequence in C. elegans embryos. A mixture comprising a construct of FIG. 2 and Example 1, Cas9 and sgRNA that would recognize the sgRNA sequences of the construct along with a target donor homology template for dpy-10 gene and sgRNA were prepared. Injections resulting in successful homologous recombination as evidenced by HR-reporter fluorescence were screened for a target site edit at the dpy-10 locus. The edit at the dpy-10 locus creates the cn64 allele. The cn64 allele was chosen due to easy visual detection of a dominant Rol phenotype upon creation of a R108C edit in one copy of the dpy-10 gene (Levy A D et al. Mol Biol Cell. 1993 August; 4(8):803-17).

Three HR-reporter construct configurations were tested. An important measure of performance is the enrichment ratio, which we defined as:

$$enrichment ratio = \frac{( percentage of target edits in flouresecent embryos )}{( percentage of target edits in all embryos )} .$$

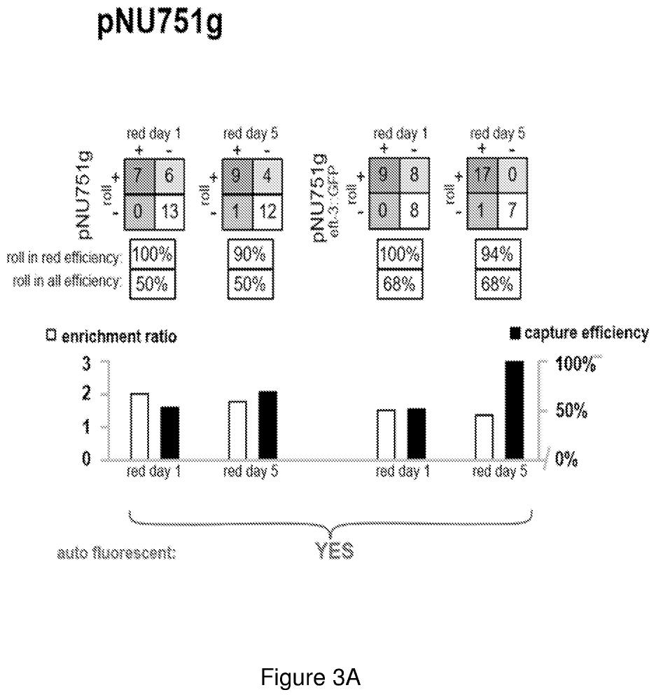

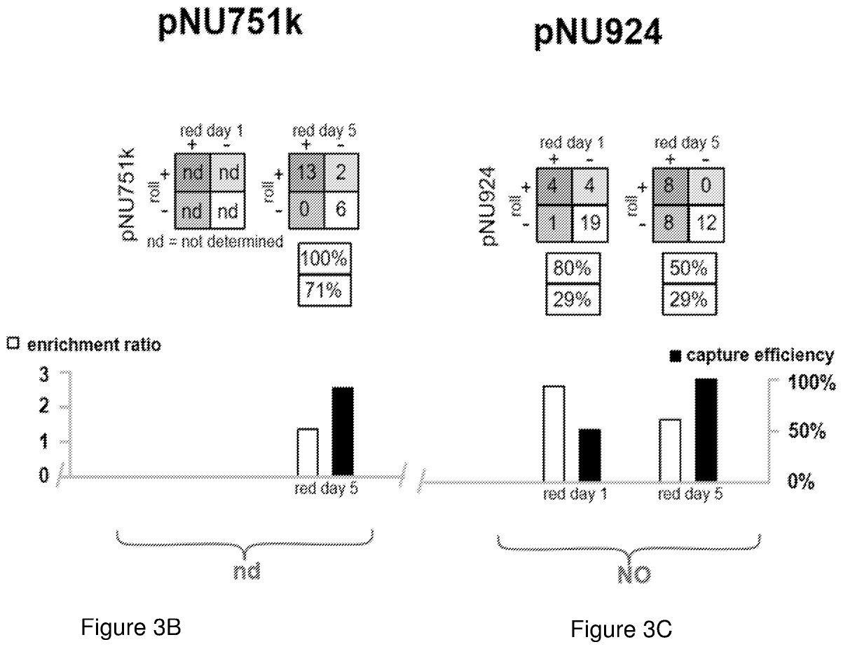

An enrichment ratio greater than 1 means that fluorescent embryos were more likely to contain target site edits than the general population of embryos. As shown in FIGS. 3A to 3C, all three reporter plasmids tested were capable of showing enrichment ratios above 1, with pNU924 demonstrating the highest enrichment of 2.75 (FIG. 3C). The timing of embryo screening is important for optimal enrichment for the target site. The pNU924 plasmid gave the best early response at 24 hours after injection in C. elegans. The pNU924 plasmid adequately forecast the R108C target site edit in 4 out of 5 (80%) red embryos observed injections at 1 day after injections. Yet at 5 days after injection, the efficiency drops to only 8 of 16 (50%) red embryos having the R108C edit. Similarly, the pNU751 plasmids showed a decrease in efficiency at day 5 vs day 1. On average, the efficiency ratio of co-correlation dropped 18% when observation of red fluorescence was performed 4 days after the first observation

Another important measure of performance is the capture efficiency, which we defined as:

$$capture efficiency = \frac{( number of target edits flouresecent embryos )}{( number of target edits in all embryos )}$$

A capture efficiency of 100% means that no target edits were lost in discarding non-fluorescent embryos. A high capture efficiency may be desired in instances where the target editing events are very rare. The higher capture efficiencies were observed by selecting fluorescent embryos at a later timepoint (i.e., 4 days). When control injections were performed with and without Cas9 nuclease for injection mixes containing either PNU751g or pNU924, only pNU924 showed absolute dependence on the presence of Cas9 nuclease. As a result, the pNU924 construct was found to be superior for avoiding autoactivation and providing the highest level of target site enrichment. In regard to reducing the number of animals in a screen, the pNU924 configuration gave the best early response at 24 hours after injection in C. elegans. The pNU924 plasmid accurately forecast the targeted edit in 4 out of 5 red embryos. While there was a decrease in co-correlation between day 1 and day 5, observations at day 5 increased the total number of edits identified. The pNU924 plasmid identified 4 of the 8 total R108C edits (50%) with red embryos at day 1. However, all of the R108C edits were identified as red embryos at day 5, demonstrating a 100% capture efficiency. Screening at day 1 for red embryos results in a greater enrichment for target site edits than screening at day 5. In contrast, screening at day 5 for red embryos results in greater capture of all the target site edits made. Depending on the difficulty of the genome edit, different screening methods may need to be applied. For instance, late screening might allow one to identify a difficult to generate target site mutation.

The pNU924 HR-reporter construct was further validated by comparing against no reporter, and the pNU344 control reporter. See FIG. 2. The pNU344 control reporter contains the same eft-3 promoter and tbb-2 utr as the pNU924 HR-reporter, but the pNU344 control reporter codes for an uninterrupted fluorescent protein and is red fluorescent without recombination. Injection mixes were created as follows: no reporter—dpy-10 donor homology template to make the R108C edit, dpy-10 sgRNA, and Cas9; pNU344—dpy-10 donor homology template to make the R108C edit, dpy-10 sgRNA, Cas9, and pNU344; pNU751g—dpy-10 donor homology template to make the R108C edit, dpy-10 sgRNA, Cas9, pNU751g and the pNU751g sgRNA; pNU924—dpy-10 donor homology template to make the R108C edit, dpy-10 sgRNA, Cas9, pNU924 and the pNU924 sgRNA. Each mix was injected into the gonads of 30 nematodes. Injected nematodes were allowed to lay progeny and their progeny were visually screened for red fluorescence and the target site dpy-10 edit. The observation of red fluorescence was found to correlate with target site dpy-10 edits. T-tests indicate significance of >0.01 for both pNU751g and pNU924 constructs when compared to the pNU344 control. When using the pNU924 HR-reporter plasmid, red fluorescence is enriched 8.4-fold for the target site edit. See FIG. 4A.

The dpy-10 phenotype observed could be due to random mutagenesis in the dpy-10 or specific repair mediated by homologous recombination. Either of these would result in the dpy-10 Rol phenotype that was observed. Sequencing of the dpy-10 gene was performed to determine the molecular nature of the mutagenesis. See FIG. 4B. From the no reporter injection set, 11 embryos (22 alleles) were isolated and sequenced. Five of the alleles (23%) showed the targeted change for dpy-10, indicating homologous recombination repair had made the desired mutation. Five of the alleles (23%) showed insertion or deletions (indels) at dpy-10, indicating incorrect repair had occurred. The remaining 12 alleles (55%) were wild-type for the dpy-10, indicating no mutagenesis had occurred. From the pNU344 fluorescent control injection set, 12 embryos (24 alleles) were isolated and sequenced. Eight of the alleles (33%) showed the targeted change for dpy-10, indicating homologous recombination repair had made the desired mutation. Eight of the alleles (33%) showed indels at dpy-10, indicating incorrect repair had occurred. The remaining 8 alleles (33%) were wild-type for the dpy-10, indicating no mutagenesis had occurred. From the pNU924 HR-reporter injection set, 12 embryos (24 alleles) were isolated and sequenced. 12 of the alleles (50%) showed the targeted change for dpy-10, indicating homologous recombination repair had made desired mutation. Five of the alleles (21%) showed indels at dpy-10, indicating incorrect repair had occurred. The remaining seven alleles (29%) were wild-type for the dpy-10 indicating no mutagenesis had occurred. This is a statistically significant improvement in the detection of desired mutagenesis (chi-squared test, p value<0.01) when compared to the baseline no marker results.

The results show a genetically encoded reporter whose fluorescence activity is triggered when recombination repair (HR) machinery is activated in the cell. Using the concept of co-CRISPR transgenesis a genome edit that creates a strong dominant phenotype is used to identify the subset of injections that are enriched for an edit at a second “target” site. Tracking the dominant roller phenotype in a co-CRISPR experiment enabled a 20-fold reduction in the number of animals that needed to be screened before finding the target edit.

The present nucleic acid constructs of FIG. 2 and Example 1 were designed to act as a homologous recombination reporter (HR-reporter) for use as a co-CRISPR reagent, wherein the functional fluorescent protein is a dominant fluorescent-phenotype marker, and the functional fluorescent protein is only expressed when successful recombination repair has removed the sequence element disrupting functional expression of the fluorescent protein. The present constructs, when introduced into the embryo are in inactive form because the construct comprises a sequence element comprising a B segment that comprises an expression disruption site, and an A′ segment that is a direct repeat of an A segment from the coding sequence of the fluorescent protein directly upstream of the sequence element, wherein the construct comprises at least one sgRNA site. Cleavage of the sgRNA site with a Cas9/sgRNA complex enables repair of the plasmid to proceed by either non-homologous end joining (NHEJ) or homologous recombination (HR). In NHEJ mediated repair, the cut ends re-ligate into forms unproductive (not fluorescent) for red fluorescent protein production. Alternatively, if HR repair has been activated, the homology of the direct repeats instructs perfect repair and an active fluorescent protein is produced. The result is HR activity in an injection is detected as a burst of fluorescent (e.g., red) protein production.

### Example 3

### Enrichment for Bi-Allelic Conversion with the HR-Reporter

Nucleic acid constructs of Example 1 may be used in methods for detecting successful homologous recombination of a second target sequence in both chromosomes in C. elegans embryos. A mixture comprising a construct of FIG. 2 and Example 1, Cas9 and sgRNA that would recognize the sgRNA sequences of the construct along with a target donor homology template for insertion of the 3×FLAG tag in the fcd-2 gene locus and fcd-2 sgRNA were prepared. A second mixture using the same target site edit as above and the standard dpy-10 sgRNA and donor homology template as co-CRISPR was prepared for comparison. Table 1 shows the screening process and results for these two mixtures. Each mixture was injected into the gonads of 30 adult nematodes. For the C. elegans injected with the HR reporter plasmid, 24-hour post-injection plates were screened for red embryos and 6/30 plates were found. Five days post-injection plates from both sets of injections were screened for the co-CRISPR phenotype. For those injected with the dpy-10 co-CRISPR, 30 plates were screened and 90 nematodes with the co-CRISPR phenotype were isolated. For those injected with the pNU924 HR-reporter, 6 plates were screened, and 15 nematodes were isolated. All of the isolated nematodes were screened by PCR for the target site edit. For those injected with the dpy-10 co-CRISPR, 90 nematodes were screened by PCR and 3 nematodes with the target site edit were identified. For those injected with the pNU924 HR-reporter plasmid, 15 nematodes were screened by PCR and 3 nematodes with the target site edit were identified. Both methods identified the same number of nematodes with the target site edit, but the HR-reporter plasmid reduced the screening effort 6-fold. The nematodes were investigated for bi-allelic conversion of the target site locus. For those injected with the dpy-10 co-CRISPR, 1 nematode with bi-allelic conversion in the F1 generation was identified. For those injected with the pNU924 HR-reporter plasmid, 3 nematodes with bi-allelic conversion in the F1 generation were identified. The enrichment for bi-allelic conversion is a significant advantage when working with animals because the homozygous mutant can be found in the first generation instead of waiting for several generations and crossing animals appropriately to make the desired homozygous line.

TABLE 1
Use of the constructs developed in Example 1 and methods of
Example 2 as Compared to Published co-CRISPR methods
for detecting homologous recombination (HR)

| | dpy-10 | Present methods |
|---|---|---|
| | co-CRISPR | and constructs |
| Total nematodes injected | 30 | 30 |
| Plates screened at 5 days | 30 | 6 |
| Nematodes isolated at 5 days | 90 | 15 |
| PCR assays performed | 90 | 15 |
| F1 nematodes with target mutation | 3 | 3 |
| Homozygous mutant F1 animals | 1 | 3 |

### Example 4

### Preparation of Present Nucleic Acid Construct for Use in Zebrafish

Nucleic acid constructs were prepared according to Example 1, except eft-3 promoter was exchanged for a zebrafish promoter, rpl3a. See FIG. 5. In swapping out the eft-3 for a zebrafish promoter, a screen of promoters was performed. For the constructs to be used in Zebrafish, it was necessary to identify a promoter with strong expression occurring early in the Zebrafish embryo shortly after fertilization. Searches of the literature (Provost E et al. Zebrafish 10, 161-169 (2013); Liu J and Lessman C. Gene Expr. Patterns 8, 237-247 (2008)) and of expression databases identified eight candidate promoters. See Table 2: All constructs were built to express mCherry under various promoters. Red fluorescence was measured after injection and given a score of no expression, low expression (+), medium expression (++), or high expression (+++).

TABLE 2
Expression time course of mCherry under early embryo promoters in
Zebrafish embryos. Red fluorescent signal was observed at 8hpi
(8 hours post injection); 12 hpi and 24 hpi.

| Promoter | Plasmid Number | Plasmid build | 8 hpi | 12 hpi | 24 hpi |
|---|---|---|---|---|---|
| tubb4b | pNU1275 | complete | no | no | no |
| tuba81 | pNU1276 | complete | + | + | + |
| tuba71 | pNU1277 | complete | no | no | no |
| rpl21 | pNU1278 | No | n/a | n/a | n/a |
| rpl13a | pNU1279 | complete | + | ++ | +++ |
| rpla10a | pNU1280 | complete | no | no | no |
| eeflg | pNU1197 | complete | no | + | no |
| tuba1a | pNU1196 | complete | no | no | no |

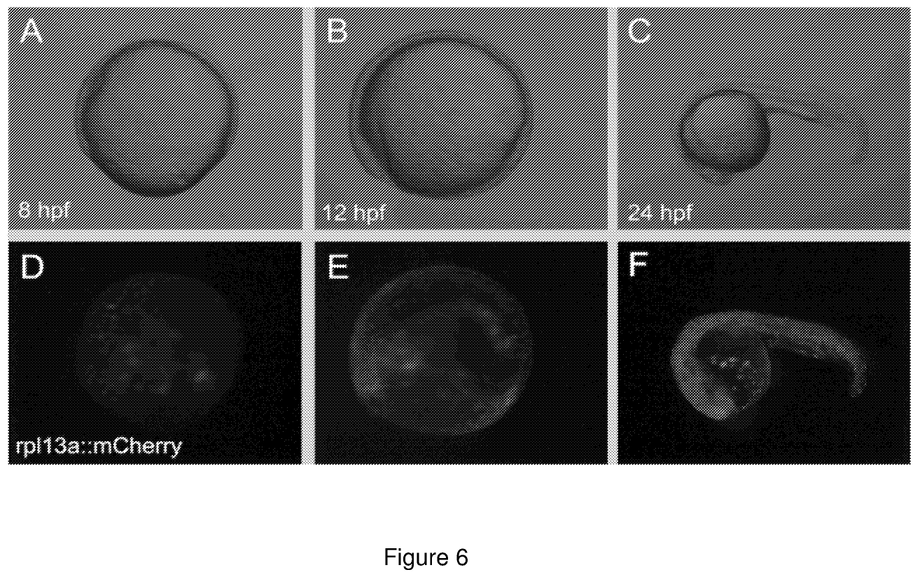

Promoters and their potential regulatory regions were amplified from zebrafish genomic DNA and cloned into a zebrafish mCherry expression vector as segments of 1000 bp in front of the gene's start codon. From the ten planned promoters, construction success was achieved for eight. After sequence confirmation, each of the 8 plasmids was injected into over 200 embryos. Fluorescent expression was monitored at 2, 4, 8 and 24 hours. Three promoters showed expression of mCherry within 24 hours after injection: the tuba8I promoter, the rpl13a promoter, and the eef1g promoter. The rpl13a promoter had the strongest expression. See FIG. 6. The eef1g promoter and the rpl13a promoter were selected for use in preparing nucleic acid constructs (zebrafish HR reporters) of the present disclosure. See FIG. 5.

Example promoter and promoter sequence for rpl13a-mCherry construct (pNU1279) is provided

SEQ ID No. 3: Zebrafish codon optimized coding
sequence for mCherry
ATGGTGAGCAAGGGCGAGGAGGATAACATGGCCATCATCAAGGAGTTCAT
GCGCTTCAAGGTGCACATGGAGGGCTCCGTGAACGGCCACGAGTTCGAGA
TCGAGGGCGAGGGCGAGGGCCGCCCCTACGAGGGCACCCAGACCGCCAAG
CTGAAGGTGACCAAGGGTGGCCCCCTGCCCTTCGCCTGGGACATCCTGTC
CCCTCAGTTCATGTACGGCTCCAAGGCCTACGTGAAGCACCCCGCCGACA
TCCCCGACTACTTGAAGCTGTCCTTCCCCGAGGGCTTCAAGTGGGAGCGC
GTGATGAACTTCGAGGACGGCGGCGTGGTGACCGTGACCCAGGACTCCTC
CCTGCAGGACGGCGAGTTCATCTACAAGGTGAAGCTGCGCGGCACCAACT
TCCCCTCCGACGGCCCCGTAATGCAGAAGAAGACCATGGGCTGGGAGGCC
TCCTCCGAGCGGATGTACCCCGAGGACGGCGCCCTGAAGGGCGAGATCAA
GCAGAGGCTGAAGCTGAAGGACGGCGGCCACTACGACGCTGAGGTCAAGA
CCACCTACAAGGCCAAGAAGCCCGTGCAGCTGCCCGGCGCCTACAACGTC
AACATCAAGTTGGACATCACCTCCCACAACGAGGACTACACCATCGTGGA
ACAGTACGAACGCGCCGAGGGCCGCCaCTCCACCGGCGGCATGGACGAGC
TGTACAAG
SEQ ID NO. 4: rpl13a promoter sequence
ggtgcatttggcaagaaacaggccgctgaggaggagatgtacttcaagtg
agtggttttgcttgagctgataattatgtaattgcttatacttgatatct
actggccattagctgagtattattgaaaaaataactgaatgtaaagcaac
ctaaaccgttacttcatgacctattctgtcattgtatttccttcacagga
gaaaagagcaagaacagctgtctgctctgagaagacaccaccaggaagag
attgaccatcacaagaaggaaatcgagagattacagcatgagatcacccg
ccacgagagcaaaatcaagaaactcaaacatgatgactgaggcattaaga
cagaaaatacaacacatgaattgtgaaactgctgaatatttgtaattgct
tatttactaaacagtgaactctgtgattatactattataaaagcatgtta
taatacagatatggttatataactgaaacaacacattgtgtattaaccca
gtgcattttccctcttttgacaataaacaagaaattgtctcgaatgtaaa
agtgtgtcttggtatcaatacgtttggtgaaagctaactattagctaaac
taactaaagctaactattggtttgagagctaaatgtatcttaactgttac
tttcagtcatataaataggttatgtcatctgaccagacaattaaaggttc
tgacaccaatgaatgacccaatattgtataaatatgagatatattaaaat
atgccgtaatgctgggtttcaggatcagattgagaaacactgctttagaa
aatgttcgagacaacacttctttattattatatttttaatattttaaagg
cgttgtagctcattggagcccagctgatggcagtagacataaataacagg
cattacaaacgtcctctgaagaacagctaatcctaacgtcatttccgatg
acgcgaaagctccgccctcgcccctgtcttttacgccaggcggccccgcg
tgtctttcttttcccacatc

To create a zebrafish HR-reporter plasmid, an A segment to be used as a direct repeat with an inserted A′ segment was selected in the zebrafish codon-optimized sequence for mCherry. The plasmid pNU1455 was created by inserting an A′ segment 23 bp in length as a direct repeat of the identified A segment into pNU1279, wherein the B segment between the direct repeat A and A′ segments comprise a sgRNA recognition site predicted to be an efficient guide for Cas9 cutting but non-native to zebrafish. See FIG. 5. The resulting intervening sgRNA site creates a frameshift leading to early termination. Because of poor performance as a HR-reporter (very low brightness) a second HR-reporter configuration (pNU1579) was created. Similar to the pNU1455 plasmid, the pNU1579 plasmid employed the use of a pair of direct repeats. Yet, the repeat length was expanded to 40 bp and the B segment was designed to encoded two sgRNA sites flanking a stop codon. Unlike the single sgRNA site in the first plasmid, both of the two new sgRNA sites were designed to leave 2 and 5 base pair overhangs on the 3′ ends of the homology arms. A third plasmid configuration (pNU1902) was made similar to pNU1579, except the homology arms contain 0 and 1 base pair overhangs after the two sgRNAs cleave the HR reporter plasmid. A fourth plasmid configuration (pNU1903) was designed similar to pNU1902 except the A and A′ segments are 42 base pairs in length. See FIG. 5.

### Example 5

### Method of Increasing Likelihood of Detecting Successful Modification of a Specific Sequence in Chromosomal DNA of Zebrafish Using CRISPR/Cas9

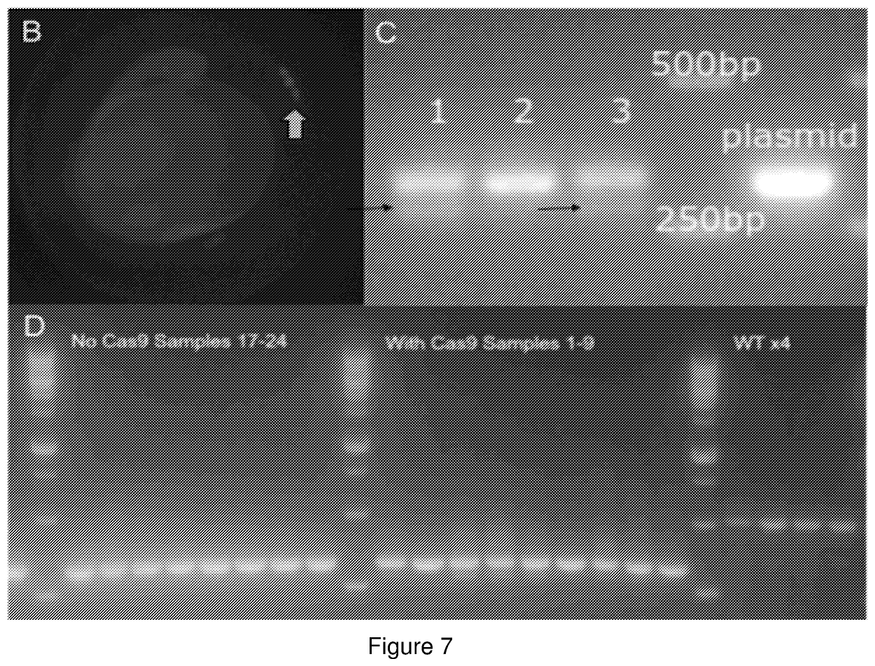

Similar to Example 2, except in Zebrafish, nucleic acid constructs were prepared according to Example 4 and used in methods for detecting successful homologous recombination of a target sequence in Zebrafish embryos. The pNU1455 plasmid construct, along with sgRNA, and Cas9 were injected into zebrafish embryos and the embryos were screened for presence of fluorescence. Red fluorescence was observed in a small subset of cells at 18 and 24 hours after injection. See FIG. 7 panel B. Because the reporter was too dim to use for visual fluorescence detection, a PCR test was used to determine if recombination occurred in the injected embryos. Primers annealing outside but amplifying across the recombination region create a PCR band of 283 bp only when recombination has occurred, otherwise a 380 bp band occurs from the unedited plasmid. See FIG. 7, panel C. The 283 bp band was DNA sequenced from 11 positive embryos and correct recombination was observed in 10 samples. To ensure the recombination PCR signal was dependent on CRISPR/Cas9-mediated transgenesis, injections were performed with and without the Cas9 protein. No recombination PCR signal was observed in the injections without Cas9 protein indicating that the recombination was dependent on Cas9 cutting of the DNA. See FIG. 7 panel D.

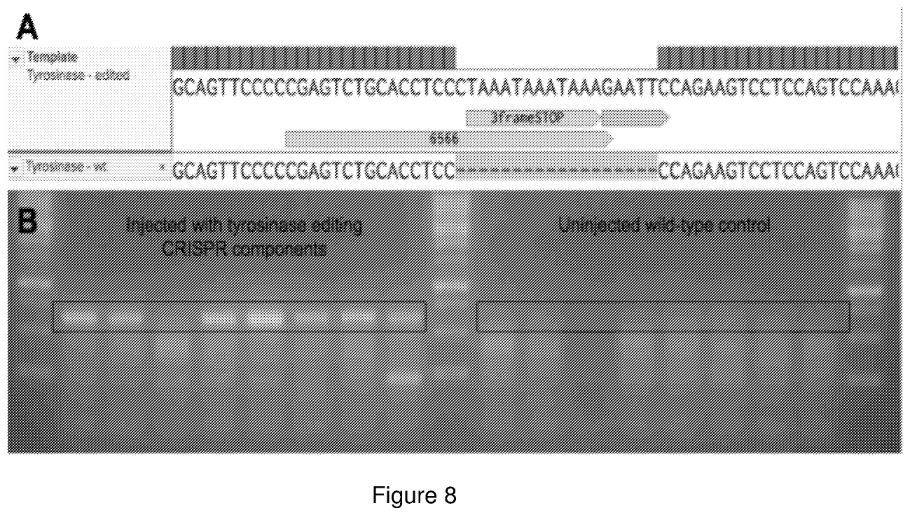

Nucleic acid constructs were prepared according to Example 4 and used in methods for detecting successful homologous recombination of a target sequence in Zebrafish embryos. A mixture comprising the a pNU1455 construct of FIG. 5 and Example 4, Cas9 and sgRNA that would recognize the sgRNA sequences of the construct along with a target donor homology template to insert a stop codon in the tyrosinase locus and sgRNA were prepared. See FIG. 8 panel A. The tyrosinase locus was chosen because a highly efficient sgRNA target site was previously identified for this locus (Chen W et al. Proc Natl Acad Sci USA. 2013 Aug. 20; 110(34):13904-9). A plasmid donor homology template was used to introduce a stop codon in the first exon of the tyrosinase gene locus (Phe27fsX3). After co-CRISPR injection, it was determined plasmid pNU1455 HR reporter inefficiently expressed the red fluorescent protein for visual sorting by red fluorescence. To determine if homologous recombination-mediated repair of the reporter had occurred, the embryos were harvested for PCR to determine if low levels of recombination could be detected. A PCR screen for HR reporter activity was designed to detect reporter editing as a 283 bp only when recombination has occurred, a 380 bp band occurs from the unedited plasmid. See FIG. 8 panel B. From the 191 embryos injected and screened, 40 embryos were identified as having the highest recombination signal. Another 40 embryos were identified as having the recombination fluorescent signal. These were then tested for the target site homologous recombination at the tyrosinase locus by PCR. The PCR assay was designed to only give amplification of a 631 bp band if the target site edit was created. In the embryos with the highest fluorescent signal, 11 embryos contained with the tyrosinase repair band of 631 bp size. In the embryos with the lowest fluorescent signal, only 3 embryos were positive for the tyrosinase repair band. The resulting increase in desired co-correlating edits in the high fluorescence signal population was determined to be statistically significant by a Fisher's Exact test. If there was no correlation, 5 of 40 embryos selected at random would be expected to exhibit the tyrosinase signal. As a result, the ability to detect high levels of HR reporter activity enables 2.2-fold enrichment in finding desired target site (tyrosinase locus) edits.

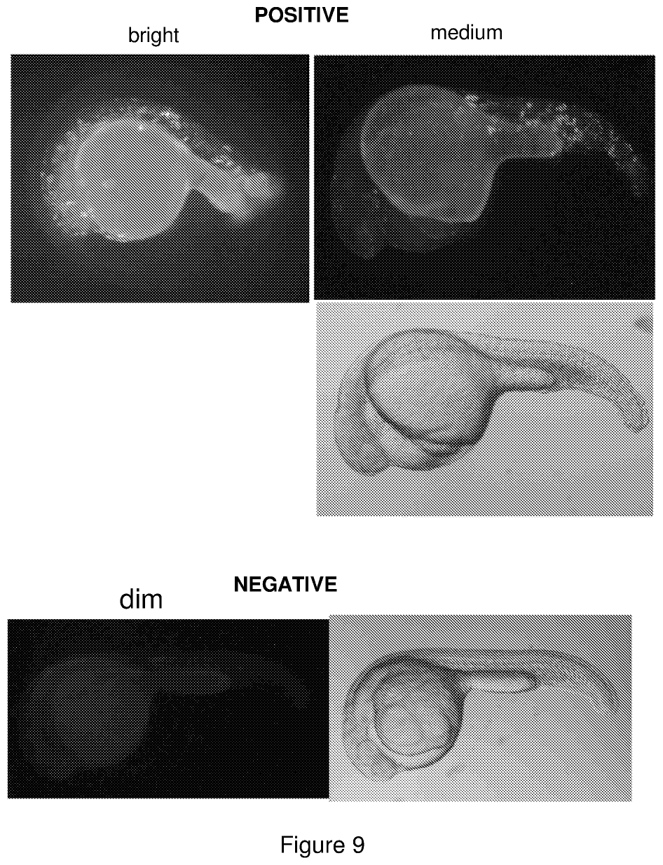

Because the pNU1455 HR-reporter was too weak for practical utilization, two other configurations of the reporter as disclosed in Example 4 (pNU1579 and pNU1902) were created and tested for capacity to be reporters of high fluorescence capacity. The pNU1579 plasmid construct was capable of exhibiting high levels of fluorescence signal. See FIG. 9. Embryos from injections were ranked and categorized into those POSITIVE for the reporter signal and those NEGATIVE for the reporter signal. A set of three independent co-CRISPR injections were performed. After reporter signal categorization, the embryos were tested by PCR for the target site edit at the tyrosinase locus. See Table 3. Statistical significance was achieved (p>0.05) for capacity of HR-reporter activation to correlate with observation of the target site edit at the tyrosinase locus. The enrichment by selecting only POSITIVE embryos averaged to 4× higher than if embryos were randomly selected.

TABLE 3
Correlation capacity in zebrafish

| | | | | Observed | Expected |
|---|---|---|---|---|---|
| | | | | tyr | tyr |
| Category | test 1 | test 2 | test 3 | edit | edit |
| POSITIVE | 7/18 | 6/36 | 17/51 | 30/105 | 21/105 |
| NEGATIVE | 3/16 | 2/32 | 0/22 | 5/70 | 14/70 |

| | |
|---|---|
| Chi Squared = 9.643 (1DF) | p = 0.0019 |

Fold enrichment = 4×

### Example 6

### Method of Increasing Likelihood of Detecting Successful Germline Genetic Modification of a Specific Sequence in Chromosomal DNA of Zebrafish

To demonstrate germline integration events are indicated by the HR-reporter, a nucleic acid reporter construct pNU1902 was prepared according to Example 4 and used to detect homologous recombination repair of a target sequence in the germline of Zebrafish. Two independent co-CRISPR injections were performed targeting two different genomic regions. Data not shown for the second genomic region but results were similar. Injection mix components for the first genomic region target are disclosed in Table 4.

TABLE 4
Reagents for germline correlation capacity
in zebrafish
Injection mix 1: STXBP1 S42P target edit

| | | Final |
|---|---|---|
| Component | Sequence | Concentrations |
| target | TAGTGGACCAGCTCAGCA | 1.5 pmol/ul ng/ul |
| sgRNA | TG | |
| | (SEQ ID NO: 5) | |
| repair | GCCCTCTGTCATGATATC | 25 ng/ul |
| DNA | AGTCATTTTGCAGCAGGA | |
| | AGGCAGCATGCGCATGCT | |
| | GAGCTGGTCCACTATCAA | |
| | AGCCTACAGAGAGAA | |
| | (SEQ ID NO: 6) | |
| HR | GCTACCATAGGCACCACG | 1.5 pmol/ul |
| reporter | AG | |
| sgRNA | (SEQ ID NO: 7) | |
| HR | pNU1902 | 50 ng/ul |
| reporter | | |
| plasmid | | |
| Cas9 | | 375 ug/ml |
| protein | | |
| Phenol red | | 0.025% |

Zebrafish embryos were injected using standard techniques with 1-2 nl of injection mix 1 to target the S42P locus of the stxbp1a gene. The injection mix was made as follows: 1.5 ul Cas9 (5 mg/ml stock solution), 1.0 ul target sgRNA (30 uM stock solution), 1 ul target repair DNA template (500 ng/ul stock solution), 1.0 ul HR reporter sgRNA (30 uM stock solution), 1 ul HR reporter plasmid pNU1902 (50 ng/ul final concentration), 1 ul 0.5% phenol red, and 13.5 ul H₂O. Injections were performed using pulled glass capillaries loaded with the injection mix. Embryos were collected after fertilization and injected immediately (within 45 minutes post fertilization). 250-300 embryos were injected.

After injection, reporter signal categorization was made. Embryos were determined to be either reporter positive (bright or medium signal) or negative (dim signal) based on red fluorescence signal. See FIG. 9. The Zebrafish were grown to adulthood. The animals that survived to adulthood were outcrossed to wild-type Zebrafish to identify those that transmit the genome edit into the next generation. The genome edit was detected in pooled embryos from the outcross by allele-specific PCR (AS-PCR). Primers were designed so that only edited sequences would produce PCR amplification products.

TABLE 5
Germline correlation capacity in zebrafish

| | stxbp1a S42P | stxbp1a S42P |
|---|---|---|
| | HR-reporter | HR-reporter |
| | negative | positive |
| Zebrafish screened | 15 | 28 |
| AS-PCR positive hits | 1 | 4 |

Of the fifteen HR-reporter negative Zebrafish tested, only 1 contained an AS-PCR positive hit indicating a genome edited line. However, of the 28 HR-reporter positive Zebrafish tested, 4 contained an AS-PCR positive hit indicating a genome edited line. This represents a two-fold enrichment for germline edits in the HR-reporter positive embryos (14%) vs the HR-reporter negative embryos (7%). This increase in genome edited germlines indicate that the HR-reporter measured in the embryo after injection can be a useful tool for identifying those embryos where genome editing of the germline is likely.

### Example 7

### Methods of Identifying Compounds that Increase Homologous Recombination Using Present HR Reporter Constructs

The HR reporter pNU1902 (See FIG. 5) was combined with locus targeting reagents (See Table 5 below) as in example 6 with targeting for restoration-of-function edits of nacre locus in a nacre −/− zebrafish strain and p53 morpholino. The p53 morpholino (Gene Tools, LLC) allows examination of effect on homologous recombination activity when p53 mediated induction of NHEJ activity is lost. Three injection mixes were made as provided in Table 5.

TABLE 5
Injection mixes demonstrating the use of the HR-reporter in identifying
compounds that increase homologous recombination.

| | | Injection mix 1 | Injection mix 2 | Injection mix 3 |
|---|---|---|---|---|
| Component | Sequence | concentration | concentration | concentration |
| target | TTGCAGTTGAACGAAGAAGG | | 1.5 uM | 1.5 uM |
| sgRNA #1 | (SEQ ID NO: 8) | | | |
| target | ATGACAGAATTAAGGAGCTG | | 1.5 uM | 1.5 uM |
| sgRNA #2 | (SEQ ID NO: 9) | | | |
| repair DNA | AATGTCTCGtttttttttCA | | 25 ng/ul | 25 ng/ul |
| | TCCTTGCAGTTGAACGAAGA | | | |
| | AGACGATTCAACAtCAACGA | | | |
| | TAGGATCAAAGAACTGGGGA | | | |
| | CTTTAATTCCCAAGTCAAA | | | |
| | TGATCCGTAAGTTT | | | |
| | (SEQ ID NO: 10) | | | |
| HR | GCTACCATAGGCACCACGAG | 1.5 uM | 1.5 uM | 1.5 uM |
| reporter | (SEQ ID NO: 7) | | | |
| sgRNA | | | | |
| HR | pNU1902 | 25 ng/ul | 25 ng/ul | 25 ng/ul |
| reporter | | | | |
| plasmid | | | | |
| Cas9 | | 375 ug/ml | 375 ug/ml | 375 ug/ml |
| protein | | | | |
| p53 | | 0.1 mM | | 0.1 mM |
| morpholino | | | | |
| Dextran | | 2.5 mg/ml | 2.5 mg/ml | 2.5 mg/ml |
| fluorescein | | | | |

Injections were performed using pulled glass capillaries loaded with one of the three injection mixes. Embryos were collected after fertilization and injected immediately (within 45 minutes post fertilization). 250-300 embryos were injected per injection mix.

After injection, embryos from all three injection mixes were analyzed using red fluorescence. Embryos were categorized as “strong”, “weak”, and “negative” based on the red fluorescence signal, See FIG. 9. Embryos in these conditions provide a measure of the levels of HR activity via the observation of fluorescent puncta made by HR reporter activity.

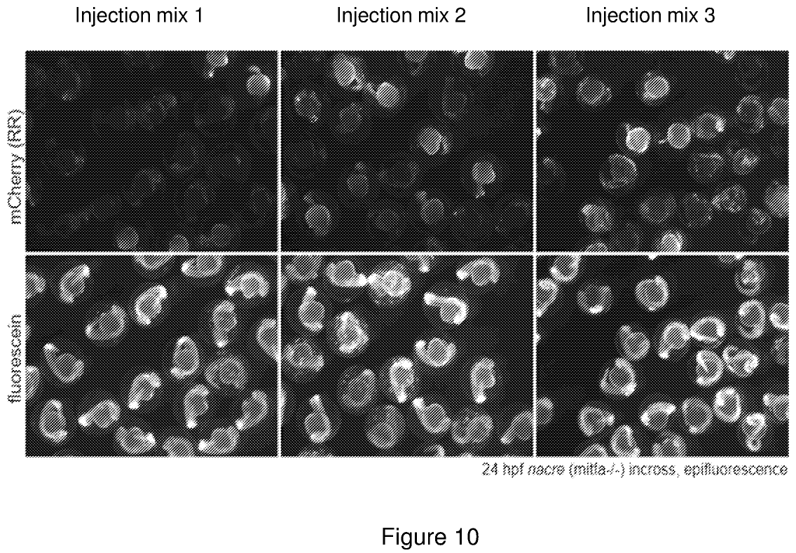

The first test condition (Injection Mix 1) was composed of reporter reagents (HR reporter sgRNA, HR reporter plasmid, and Cas9) with p53 morpholino. This test condition lacks the nacre target reagents (nacre repair DNA and nacre sgRNAs). The lack of nacre sgRNA precludes genomic cutting and HR reporter activity is highly attenuated. The capacity to generate bright puncta (“strong”) is very limited and very few embryos generate bright puncta. See FIG. 10. Quantified across the injected clutch, bright puncta were a rare occurrence (0.8%) in the “strong” category of injected embryos. See Table 6. In the “weak” category of injected embryos, more low intensity puncta were observed (31.6%). In the “negative” category, the lacking HR reporter activity resulted in 67.6% of embryos with no detectable fluorescent puncta.

TABLE 6
Quantified results for p53 knockdown induction of HR reporter activity

| HR | | # of | # of | % of |
|---|---|---|---|---|
| reporter | experimental | normal | embryos | normal |
| Strength | group | embryos | observed | embryos |
| strong | Injection | 250 | 2 | 0.8 |
| weak | mix | | 79 | 31.6 |
| negative | 1 | | 169 | 67.6 |
| strong | Injection | 203 | 18 | 8.9 |
| weak | mix | | 110 | 54.2 |
| negative | 2 | | 75 | 36.9 |
| strong | Injection | 255 | 57 | 22.4 |
| weak | mix | | 164 | 64.3 |
| negative | 3 | | 34 | 13.3 |

In the second and third test conditions the levels of “strong” reporter category increase. In test condition 2 (Injection Mix 2) the reagents for cutting the target locus (nacre repair DNA and nacre sgRNAs) are present but the p53 morpholino is absent. In this test condition, many more bright puncta are generated in the “strong” category (8.9%) compared with Injection Mix 1 (0.8%). The activity in the “weak” category has marginally increased from 31.6% to 54.2%. In the third test condition (Injection Mix 3), reagents for cutting the target locus and the p53 morpholino for knocking down in p53 expression are both present. The bright puncta of the “strong” category are now significantly higher (22.4%) and the “weak” category has increased to 64.3%.

Comparison of test 1 to test 3 shows the effect of cutting genomic DNA on activation of the HR reporter. The bright puncta of the “strong” category have increased 28× and the faint puncta of the “weak” category have increased 2×. These results indicate the cutting of genomic DNA is a strong activator of homologous recombination activity in the embryo.

Comparison of test 2 to test 3 measures the effect of down regulation of NHEJ activity on the activation of the HR reporter. The inclusion of p53 morpholino leads to an enhancement of the HR reporter activity. The bright puncta of the “strong” category have increased 2.5× and the faint puncta of the “weak” category have increased 1.7×. These results indicate the disruption of NHEJ by p53 morpholino is an effective activator of homologous recombination activity in the embryo. Activity of the HR reporter was linked to genome editing of the cells by observing the repair of the nacre locus by homologous recombination and the rescue of the nacre pigment loss phenotype. In the embryos with “strong” reporter signal observed, 1.5% of the embryos had nacre phenotype rescue (homologous recombination in the genome) when the p53 morpholino was not used. This is in contrast with 4.7% of the embryos with nacre phenotype rescue (homologous recombination in the genome) in the “strong” reporter signal when the p53 morpholino was used. More genome homologous recombination was observed with the use of the p53 morpholino which matched with an increase in the HR-reporter signal. Further, this result indicates that the HR-reporter can be used to identify compounds that increase homologous recombination.

The HR-reporter is used to screen for compounds causing induction of native homologous repair processes. Embryos are injected with the compound and the HR-reporter. Alternatively, a stable line containing genome integration of the reporter is used along with incubating the embryos in the compounds. Observation of an increased fluorescence signal in the embryo is an indication that the compound has an effect on inducing homologous recombination.

We claim:

1. A nucleic acid construct comprising a gene for a mutated fluorescent protein, wherein the gene comprises;
a sequence element that disrupts expression of a functional fluorescent protein and wherein the sequence element is removed with successful homologous recombination in a host cell restoring the functional fluorescent protein, wherein the sequence element comprises;
a B segment and an A′ segment, wherein the B segment comprises an expression disruption site; and,
the A′ segment comprises a direct repeat of an A segment immediately upstream of the B segment, wherein the A segment comprises a portion of a coding sequence of the fluorescent protein from 15 base pairs to 3000 base pairs in length.

2. The construct of claim 1, wherein a translated sequence of the mutated fluorescent protein comprising the sequence element is truncated, destabilized, inactive, or produces a fluorescent signal quantitatively distinguished from a translated sequence of the functional fluorescent protein that does not comprise the sequence element.

3. The construct of claim 2, wherein quantitatively distinguished signal comprises intensity of signal or emission signal wavelength.

4. The construct of claim 1, wherein the A and A′ segments are 20 base pairs to 1000 base pairs in length.

5. The construct of claim 1, further comprising one or more nuclease cleavage sites at or flanking the sequence element site.

6. The construct of claim 5, wherein the nuclease cleavage site is recognized by nucleases selected from Cas9, Cpf1, TALen, Zinc-finger, I-Sce I, Endo.sce, HO, I-Ceu I, I-Chu I, I-Cre I, I-Csm I, I-Dir I, I-DMO I, I-Flmu I, I-Flmu II, I-Ppo I, I-Sce III, I-Sce IV, I-Tev I, I-Tev II, I-Tev III, PI-Mle I, PI-Mtu I, PI-Psp I, PI-Tli I, PI-Tli II or PI-Sce V.

7. The construct of claim 1, further comprising two nuclease cleavage sites, one located at or near an interface between the A segment and B segment, and a second located at or near an interface between the B segment and A′ segment.

8. The construct of claim 1, further comprising one or more specific guide RNA (sgRNA) recognition sequences.

9. The construct of claim 8, wherein at least one of the sgRNA recognition sequences is located at or near the interface between the A segment and B segment.

10. The construct of claim 8, further comprising a second sgRNA recognition sequence located at or near the interface between the B segment and A′ segment.

11. The construct of claim 5, wherein the nuclease cleave site is recognized by an RNA-guided endonuclease.

12. The construct of claim 1, further comprising one of more homology regions, wherein the regions are homologous with a region of a genome of the host cell for integration of the mutated fluorescent protein coding sequence into the genome of the host cell.

13. The construct of claim 1, wherein the construct is not integrated into a genome of the host cell and does not comprise one or more homology regions that are homologous with a region of the genome of the host cell.

14. The construct of claim 1, further comprising a genomic insertion sequence operably linked to the gene that expresses the mutated fluorescent protein, wherein the genomic insertion sequence and gene are located between two homology regions that are homologous with a region of a genome of the host cell.

15. The construct of claim 14, wherein the genomic insertion sequence is an ortholog gene, or fragment thereof, of the host cell.

16. The construct of claim 14, wherein the genomic insertion sequence, when the construct is added to the host cell, provides site directed mutagenesis of a host cell gene.

17. The construct of claim 14, wherein the genomic insertion sequence, when the construct is added to the host cell, replaces a host ortholog at a native locus.

18. The construct of claim 14, wherein genomic insertion sequence, when the construct is added to the host cell, disrupts expression of a host cell gene.

19. The construct of claim 1, wherein the expression disruption site comprises a stop codon, frameshift codon, one or more point mutations, one or more destabilizing codons, protease site, sequence-encoded degradation signal, or a self cleaving peptide sequence.

20. The construct of claim 1, wherein the B segment comprises a heterologous sequence that is stuffer nucleic acid, coding sequence for a fluorescent protein, or a non-coding sequence.

21. The construct of claim 1, wherein the sequence element comprises one or more sgRNA sequences, an A′ segment 20 base pairs to 600 base pairs in length, a B segment comprising a stop codon, and an endonuclease cleavage at an interface between the A and B segments.

22. The construct of claim 1, wherein the host cell is an embryo cell.

23. The construct of claim 1, wherein the host cell is an embryo cell of a mammal, a zebrafish, a livestock animal, a farm animal, a nematode, or an avian.

24. The construct of claim 1, wherein the host cell is a plant cell, a bacterial cell, or a yeast cell.

25. The construct of claim 24, wherein the plant cell is a food crop plant or an agriculture plant crop.

26. A method of increasing likelihood of detecting successful modification of a specific sequence in chromosomal DNA of a host cell via homologous recombination, comprising:
introducing a construct of claim 1 into the host cell;
introducing gene editing reagents into the host cell comprising a donor target sequence; and,
observing a desired detectable marker expressed from the construct in those host cells with successful homologous recombination gene editing.

27. The method of claim 26, wherein the gene editing reagents comprise one of Cas9, Cpf1, TALen, Zinc-finger, I-Sce I, Endo.sce, HO, I-Ceu I, I-Chu I, I-Cre I, I-Csm I, I-Dir I, I-DMO I, I-Flmu I, I-Flmu II, I-Ppo I, I-Sce III, I-Sce IV, I-Tev I, I-Tev II, I-Tev III, PI-Mle I, PI-Mtu I, PI-Psp I, PI-Tli I, PI-Tli II or PI-Sce V nucleases.

28. The method of claim 26, wherein the sequence element of the construct comprises all or part of at least one sgRNA recognition sequence.

29. The method of claim 26, wherein the gene editing reagents comprise a genomic integration sequence flanked by homology regions, wherein the regions are homologous with a region of a genome of the host cell and an endonuclease.

30. The method of claim 26, wherein the gene editing reagents comprises a sgRNA/Cas9 complex wherein the sgRNA recognizes a sgRNA recognition site on the construct.

31. The method of claim 26, wherein the fluorescent protein coding sequence is codon optimized for the host cell.

32. A method of increasing likelihood of detecting successful modification of a specific sequence in chromosomal DNA of a host cell via homologous recombination, comprising:
introducing a construct of claim 1 into the host cell;
introducing gene editing reagents into the host cell comprising:
Cas9 complexed with a sgRNA that binds a sgRNA recognition site on the construct;
Cas9 complexed with a sgRNA that binds a sgRNA recognition site on the chromosomal DNA; and,
a genomic insertion sequence located between two homology regions that are homologous with a region of the chromosomal DNA of the host cell; and,
observing a desired detectable marker expressed from the construct in those host cells with successful homologous recombination gene editing.

33. An expression plasmid comprising SEQ ID NO. 1, SEQ ID NO. 2, SEQ ID NO. 3 or SEQ ID NO 4.

34. Use of a promoter for expression of a gene in a zebrafish embryo, comprising contacting the zebrafish embryo with an expression vector comprising the promotor rpl13a.

35. A method of identifying test compounds that increase homologous recombination in a host cell, comprising:
introducing a construct of claim 1 into the host cell;
introducing gene editing reagents into the host cell;
introducing a test compound into the host cell;
observing a desired detectable marker expressed from the construct in those host cells with successful homologous recombination gene editing; and,
comparing the desired detectable signal to a control wherein the control is a host cell without a test compound and selecting those test compounds that produced an increased detectable signal in a host cell as compared to the control.

36. The method of claim 35, wherein the gene editing reagents further comprise a donor target sequence.

37. The method of claim 35, wherein the gene editing reagents induce double strand DNA breaks.

38. The method of claim 35, wherein test compounds are selected from a therapeutic agent, a drug, a drug candidate, a nutritional supplemental, vitamin or food stuff.


SEQUENCE LISTING (10 sequences)

SEQ ID NO: 1 - DNA, 869 bp, Artificial Sequence
  C. elegans codon-optimized mCherry sequence
atggtctccaagggagaggaggacaacatggccatcatcaaggagttcatgcgtttcaaggtccacatggagggatccgtcaacggacacgagttcgagatcgagggagagggagagggacgtccatacgagggaacccaaaccgccaagctcaaggtcaccaaggtaagtttaaacatatatatactaactaaggaggccctgattatttaaattttcagggaggacccctcccttttgcttgggatattctttccccccaattcatgtacggatctaaagcctacgtcaagcacccagccgacatcccagactacctcaagctctccttcccagagggattcaagtgggagcgtgtcatgaacttcgaggacggaggagtcgtcaccgtcacccaagactcctccctccaagacggagagttcatctataaggtaagtttaaacagttcggcgcgccctaaccatacatatttaaattttcaggtcaagctccgtggaaccaacttcccatccgacggaccagtcatgcaaaagaagaccatgggatgggaggcctcctccgagcgtatgtacccagaggacggagccctcaagggagagatcaagcaacgtctcaagctcaaggtaagtttaaacatgattttactaactaactaatctgatttaaattttcaggacggaggacactacgacgccgaggtcaagaccacctacaaggccaagaagccagtccaactcccaggagcctacaacgtcaacatcaagctcgacatcacctcccacaacgaggactacaccatcgtcgagcaatacgagcgtgccgagggacgtcactccaccggaggaatggacgagctctacaagtaa

SEQ ID NO: 2 - DNA, 573 bp, Artificial Sequence
  eft-3 gene promoter
tgtttctgttaaattaatgaatttttcataaaataaagacattatacaatataaaaatgaagaatttattgaaaataaactgccagagagaaaaagtatgcaacactcccgccgagagtgtttgaaatggtgtacggtacattttcgtgctaggagttagatgtgcaggcagcaacgagagggggagagatttttttgggccttgtgaaattaacgtgagttttctggtcatctgactaatcatgttggttttttgttggtttattttgtttttatctttgtttttatccagattaggaaatttaaattttatgaatttataatgaggtcaaacattcagtcccagcgtttttcctgttctcactgtttagtcgaatttttattttaggctttcaacaaatgttctaactgtcttatttgtgacctcactttttatatttttttaatttttaaaaatattagaagtttctaggataattttttcgacttttattctctctaccgtccgcactcttcttacttttaaattaaattgtttttttttcagttgggaaacactttgctcactccgta

SEQ ID NO: 3 - DNA, 708 bp, Artificial Sequence
  Zebrafish codon optimized coding sequence for mCherry
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 4 - DNA, 1020 bp, Artificial Sequence
  rpl13a promoter sequence
ggtgcatttggcaagaaacaggccgctgaggaggagatgtacttcaagtgagtggttttgcttgagctgataattatgtaattgcttatacttgatatctactggccattagctgagtattattgaaaaaataactgaatgtaaagcaacctaaaccgttacttcatgacctattctgtcattgtatttccttcacaggagaaaagagcaagaacagctgtctgctctgagaagacaccaccaggaagagattgaccatcacaagaaggaaatcgagagattacagcatgagatcacccgccacgagagcaaaatcaagaaactcaaacatgatgactgaggcattaagacagaaaatacaacacatgaattgtgaaactgctgaatatttgtaattgcttatttactaaacagtgaactctgtgattatactattataaaagcatgttataatacagatatggttatataactgaaacaacacattgtgtattaacccagtgcattttccctcttttgacaataaacaagaaattgtctcgaatgtaaaagtgtgtcttggtatcaatacgtttggtgaaagctaactattagctaaactaactaaagctaactattggtttgagagctaaatgtatcttaactgttactttcagtcatataaataggttatgtcatctgaccagacaattaaaggttctgacaccaatgaatgacccaatattgtataaatatgagatatattaaaatatgccgtaatgctgggtttcaggatcagattgagaaacactgctttagaaaatgttcgagacaacacttctttattattatatttttaatattttaaaggcgttgtagctcattggagcccagctgatggcagtagacataaataacaggcattacaaacgtcctctgaagaacagctaatcctaacgtcatttccgatgacgcgaaagctccgccctcgcccctgtcttttacgccaggcggccccgcgtgtctttcttttcccacatc

SEQ ID NO: 5 - DNA, 20 bp, Artificial Sequence
  STXBP1 S42P target sgRNA
tagtggaccagctcagcatg

SEQ ID NO: 6 - DNA, 87 bp, Artificial Sequence
  STXBP1 S42P repair DNA
gccctctgtcatgatatcagtcattttgcagcaggaaggcagcatgcgcatgctgagctggtccactatcaaagcctacagagagaa

SEQ ID NO: 7 - DNA, 20 bp, Artificial Sequence
  STXBP1 S42P HR reporter sgRNA
gctaccataggcaccacgag

SEQ ID NO: 8 - DNA, 20 bp, Artificial Sequence
  target sgRNA #1
ttgcagttgaacgaagaagg

SEQ ID NO: 9 - DNA, 20 bp, Artificial Sequence
  target sgRNA #2
atgacagaattaaggagctg

SEQ ID NO: 10 - DNA, 113 bp, Artificial Sequence
  repair DNA
aatgtctcgtttttttttcatccttgcagttgaacgaagaagacgattcaacatcaacgataggatcaaagaactggggactttaattcccaagtcaaatgatccgtaagttt